(19) United States
(12) Patent Application Publication
(10) Pub. No.: US 2025/0336485 A1
(43) Pub. Date: Oct. 30, 2025

(54) SYSTEM AND METHOD FOR PREDICTING ANTIOXIDANT SYNERGISM

(71) Applicant: Clemson University Research Foundation, Clemson, SC (US)

(72) Inventors: Carlos D Garcia, Clemson, SC (US); Lucas De Brito Ayres, Clemson, SC (US); Daniel Whitehead, Clemson, SC (US)

(73) Assignee: Clemson University Research Foundation, Clemson, SC (US)

(21) Appl. No.: 19/188,803

(22) Filed: Apr. 24, 2025

Related U.S. Application Data
(60) Provisional application No. 63/638,270, filed on Apr. 24, 2024.

(51) Int. Cl. G16C 20/30; G16C 20/70

(52) U.S. Cl. CPC G16C 20/30; G16C 20/70

(57) ABSTRACT

A computerized system for prediction of antioxidant mixtures that is capable of receiving an initial dataset of a first set of deep eutectic solvents, create a predictive model according to the initial dataset; receiving an enhancement dataset and/or a second set of experimental deep eutectic solvents, modify the predictive model according to the enhancement dataset; modify the predictive model according to a comparison of a performance of the predictive model and a test dataset; generate functional deep eutectic solvents according to the predictive model, display the resulting mixtures, the resulting mixtures being DES integrating antioxidants mixtures, and, the resulting mixtures display improved antioxidant capabilities with respect to the corresponding (non-DES forms of the same) antioxidants.

### CROSS-REFERENCE TO RELATED APPLICATIONS

This application claims priority to U.S. Provisional Patent Application No. 63/638,270, titled SYSTEM AND METHOD FOR PREDICTING ANTIOXIDANT SYNERGISM filed Apr. 24, 2024, which is hereby incorporated by reference in its entirety.

### BACKGROUND

### 1) Field of the Invention

This system is directed to the use of system for training and using artificial intelligence approach that can include providing deep learning to predict the type and degree of interaction (e.g., synergistic, additive, and antagonistic) of known mixtures as well as to provide for new antioxidant combinations for certain applications.

### 2) Description of the Related Art

The prediction of interaction (e.g., synergistic, additive, and antagonistic) of known mixtures is useful in many areas. For example, a system that can predict synergistic mixtures of antioxidants, simultaneously accounting for multiple chemical properties of the antioxidants, numerous variables related to the sample, and multiple environmental factors would be desirable.

Antioxidants can be compounds that may inhibit or delay oxidation processes in various substances. In biological systems, antioxidants can neutralize free radicals and other reactive oxygen species that may cause cellular damage. These molecules can be found naturally in many foods, particularly fruits and vegetables, or can be synthesized for use in food preservation, cosmetics, and pharmaceuticals. Antioxidants may function through different mechanisms, including scavenging free radicals by donating electrons or hydrogen atoms, chelating metal ions that can catalyze oxidative reactions, quenching singlet oxygen species and breaking oxidative chain reactions. Some common examples of natural antioxidants include vitamins C and E, beta-carotene, flavonoids, and polyphenols. In food science and technology, antioxidants may be used to prevent or slow down the oxidation of fats and oils, which can lead to rancidity and off-flavors. In some cases, combinations of different antioxidants can work synergistically, providing enhanced protection against oxidation compared to individual antioxidants used alone. The effectiveness of antioxidants can vary depending on factors such as concentration, temperature, pH, and the presence of other compounds in the matrix. Understanding these interactions and predicting synergistic effects between different antioxidants may be valuable for optimizing their use in various applications. Predicting synergistic effects between different antioxidants may also be valuable in pharmaceutical formulations, cosmetic product development, packaging materials design, and industrial lubricant manufacturing to enhance stability and extend shelf life of various products.

Lipid oxidation is a major issue affecting products containing unsaturated fatty acids, leading to the formation of low molecular-weight species with diverse functional groups that impart off-odors and off-flavors. Chemically, the oxidation of lipids is a dynamic process that ultimately leads to the formation of volatile compounds (carboxylic acids, aldehydes, and ketones) that impart unpleasant flavors and decrease the overall quality of food (appearance, texture, etc.). Besides the economic losses, rancid food can also negatively affect the health of consumers.

Aiming to control this process, antioxidants are commonly added to these products, often deployed as combinations of two or more compounds, a strategy that allows lowering the amount used and/or boosting the total antioxidant capacity of the formulation. While this approach allows minimizing the potential organoleptic and toxic effects of these compounds, predicting how these mixtures of antioxidants will behave has traditionally been one of the most challenging tasks, often leading to simple additive, antagonistic, or synergistic effects. Some research subscribes to the idea that synergistic interactions between antioxidants simply require their interaction by a combination of π-π stacking and hydrogen-bonds, predicting the intricate interplay of variables involved in these interactions remains a significant scientific challenge.

Approaches to understand these interactions have been predominantly empirically driven, but thus far inefficient low throughput endeavor and unable to account for the complexity and multifaceted nature of antioxidant responses.

Lipid oxidation, again a major issue affecting products containing unsaturated fatty acids, impact, for instance, cosmetics, vegetable oils, seafood, processed meat, and animal feed. The oxidative deterioration of these samples can occur via chemical, thermal, enzymatic, and/or photocatalytic mechanisms. Among these, auto-oxidation (spontaneously initiated in the presence of atmospheric oxygen) is the least selective and probably one of the most difficult to control. Among other targets, the oxidation of lipids leads to the formation of low molecular-weight species with diverse functional groups (carboxylic acids, aldehydes, and ketones) that impart off-odors and off-flavors. This process is also known as rancidity and can not only impart an unpleasant taste but also diminish the nutritional value and the overall quality of the sample, which may ultimately impact the health of the end consumer. Moreover, the oxidation of lipid-based foods also contributes to the shorter shelf-life of these products, resulting in considerable economic losses in all segments of the supply chain.

Therefore, it is critical to develop strategies to mitigate or prevent lipid oxidation in foods. For this purpose, the use of antioxidants has proven to be one of the most effective and frequently adopted methods, a strategy that has been also extended to pharmaceuticals, cosmetics as well as nutraceutical products. Although these antioxidants are derived from natural (e.g., tocopherols, phenolic acids, polyphenols, and ascorbic acid) or synthetic sources, they offer different mechanisms of action and allow targeting the reaction at different stages, from scavenging free radicals, to quenching triplet oxygen, to chelating metal cations. Regardless of the mechanism of action, antioxidants are normally deployed as combinations of two or more compounds, a strategy that allows lowering the amount used while boosting the total antioxidant capacity of the formulation. While this approach allows minimizing the potential organoleptic and toxic effects of these compounds, predicting how these mixtures of antioxidants will behave has traditionally been one of the most challenging tasks, often leading to simple additive (even antagonistic) effects, instead of the desired synergistic response.

Although the interaction between some classes of antioxidants is well known for specific samples, there is a current need for a strategy that could enable broader and rational predictions related to the antioxidant capacity of mixtures.

Approaches to understand these interactions have been predominantly empirically driven, where the total antioxidant effectiveness is assessed by using assays such as total oxidation index (TOTOX), thiobarbituric acid reactive substances (TBARS), peroxide value (PV), p-anisidine test, ferric reducing antioxidant power (FRAP), or DP PH scavenging. The gathered experimental data can be then analyzed as a function of the composition of the antioxidant mixture through the use of standard methods such as isobole diagrams, response curves or interaction index parameters. Albeit effective for simple experimental designs, these one-dimensional methods often hinder the evaluation of non-linear interactions due to the complexity and multifaceted nature of antioxidant responses, which are often affected by several factors such as their mechanism of action, structural properties, and matrix effects.

According to their operating mechanism, antioxidants can be classified into primary or secondary antioxidants. Primary antioxidants such as butylhydroxyanisol (BHA), butylhydroxytoluene (BHT), and propyl gallate (PG) are able to react with free radicals, quenching the propagation phase of the oxidation reaction. Secondary antioxidants decompose hydroperoxides and prevent chain branching of photochemical reactions. Owing to their suspected action as carcinogens, there is growing interest in finding new antioxidants or combinations of antioxidants that can maintain effectiveness at much lower concentrations. Unfortunately, as previously noted, predicting the behavior for these mixtures of antioxidants has traditionally been one of the most challenging tasks, often leading to simple additive (or even antagonistic) effects, instead of the desired synergistic response. Among other reasons for this gap in knowledge are the use of traditional assays and standard analysis methods such as isobole diagrams, response curves or interaction index parameters, that are tedious, hinder the evaluation of nonlinear interactions, and are often affected by factors outside the experimental design.

What is needed is an accurate predictive model for combinations that do not rely upon trial-and-error methods, traditionally used when seeking effective mixtures of compounds.

Recently, there has been a significant increase in scientific research featuring Deep Eutectic Solvents (DES). These solvents were initially described as a distinct group of liquids found within plant tissues, displaying a significant role in their biochemistry, especially in the transport of compounds with medium polarity. After substantial research efforts, it is now known that DES are formed by precise combinations of a few (typically two or three) components, usually in solid state that, upon heating, result in a substance with a significantly lower melting point than the individual components (eutectic point depression). Out of those, perhaps the most interesting combinations are those considered stable DES, which remain liquid for at least a week when stored at room temperature. Many of these novel solvents, belonging to a sub-class formed by natural components (NADES), feature significant advantages over traditional organic solvents, ionic liquids, and conventional DES, including the low toxicity of their components, which are primarily natural molecules such as sugars, amino acids alcohols, and carboxylic acids.

It would be desirable for the broad range of physicochemical attributes inherent to the structure of those natural components to be should directed at the design of DES/NADES with adjustable properties such as conductivity, melting point, stability, polarity, and viscosity. However, the multifaceted properties of each component (e.g., hydrogen bond donor count, hydrogen bond acceptor count, molecular weight, surface area, melting point, hydrophobicity, etc.) pose considerable challenges to the rational development of DES/NADES with specific characteristics. While traditional approaches can explain a handful of properties of DES/NADES, they require specialized knowledge, they are not yet able to make statistically validated predictions of new mixtures, nor they provide rational guidelines to understand the behavior of DES/NADES broadly. As a result, the development of new DES/NADES is today almost exclusively done by trial-and-error and often derived from known mixtures, reflecting the complexity of this problem. Among the most important DES/NADES are functional DES/NADES, those that incorporate specific molecules in their structure and that are particularly suited to perform a specific function.

Therefore, it is an object of the present system to provide advanced AI models based on molecular fingerprints and chemical descriptors to predict synergistic mixtures of antioxidants, simultaneously accounting for multiple chemical properties of the antioxidants, numerous variables related to the sample, and multiple environmental factors.

It is another object of the present system to develop functional deep eutectic solvent (f_{DES}), integrating—for example but not exclusively-synergistic mixtures of antioxidants in their structure.

It is another object of the present system to provide results that can have experimentally verified predictions of the system using model compounds (such as oleic acid), real samples of fats (such as lard, tallow or chicken) and oils (such as soybean, rapeseed or olive).

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

The present invention provides a computerized system and process for predicting synergistic antioxidant mixtures using artificial intelligence. The system comprises an artificial neural network model that can be fine-tuned on deep eutectic solvent (DES) and natural deep eutectic solvent (NADES) data to create and enhance a predictive model for antioxidant mixtures. The invention also encompasses a foundational general chemistry model divided into antioxidant regressors that are selected, fine-tuned with benchtop data, and blended with experimental chemistry data to predict synergistic antioxidant combinations. Additionally, the invention includes a computerized process for training a machine learning model on an antioxidant mixture database, evaluating and fine-tuning the model, and outputting predicted antioxidant mixtures with associated confidence indices.

### BRIEF DESCRIPTION OF FIGURES

The construction designed to carry out the invention will hereinafter be described, together with other features thereof. The invention will be more readily understood from a reading of the following specification and by reference to the accompanying drawings forming a part thereof, wherein an example of the invention is shown and wherein:

FIG. 1A is a diagram of aspects of the system.

FIG. 1B is a diagram of aspects of the system.

FIG. 1C are results from aspects of the system.

FIG. 1D is a phase diagram of two components.

FIG. 1E are results from aspects of the system.

FIGS. 2A through 2D are results from aspects of the system.

FIGS. 3A through 3D are results from aspects of the system.

FIGS. 4A through 4B are results from aspects of the system.

FIGS. 5A through 5B are results from aspects of the system.

FIGS. 6A through 6B are results from aspects of the system.

FIGS. 7A through 7D are results from aspects of the system.

FIG. 8 is a diagram of aspects of the system.

FIG. 9 is a diagram of aspects of the system.

FIG. 10 is a diagram of aspects of the system.

FIG. 11 are results from aspects of the system.

FIG. 12 are results from aspects of the system.

FIG. 13 is an image of validation and testing result from aspects the system.

FIG. 14 are results from aspects of the system.

FIG. 15 is a diagram of aspects of the system.

FIG. 16 are results from aspects of the system.

### DETAILED DESCRIPTION

The following description sets forth exemplary aspects of the present disclosure. It should be recognized, however, that such a description is not intended as a limitation on the scope of the present disclosure. Rather, the description also encompasses combinations and modifications to those exemplary aspects described herein.

This system may be directed towards the development of “functional DES”-deep eutectic solvents formed with compounds that provide inherent advantages to the chemical functionality of the selected components. In some aspects, functional DES may incorporate synergistic mixtures of antioxidants within their structure. However, the concept may extend beyond antioxidants to include synergistic combinations of other biologically active compounds. For example, functional DES may be formed using mixtures of antibiotics or combinations of antibiotics with natural products.

The use of DES as complexes that enhance the biological activity of their component compounds represents a novel approach. By carefully selecting and combining active ingredients into a DES formulation, it may be possible to create systems with improved functionality compared to the individual components alone. The unique physicochemical properties of DES, such as low melting points and high solubilizing capacity, may contribute to enhancing the bioavailability and efficacy of the incorporated active compounds.

The system can include an artificial neural network model fine-tuned on DES and natural deep eutectic solvent NADES data to create and enhance a predictive model for antioxidant mixtures. In some aspects, the system may combine the artificial neural network model with a tabular model to enhance predictive capabilities for antioxidant mixtures. The tabular model may process structured data related to chemical properties, experimental conditions, and environmental factors in a complementary manner to the neural network. The system may utilize the artificial neural network to extract complex patterns and relationships from the DES and NADES data, while the tabular model may handle more straightforward numerical and categorical features. This hybrid approach may allow for efficient processing of both unstructured molecular data and structured experimental parameters. The system may incorporate a feature importance analysis to identify which inputs from the tabular data and neural network contribute most significantly to the predictions. This information may be used to refine the model and provide insights into the key factors influencing antioxidant synergism.

In some cases, the system may employ a multi-task learning framework where the neural network and tabular model are trained simultaneously on related tasks, such as predicting both synergistic effects and physicochemical properties of the antioxidant mixtures. This approach may allow the models to share information and improve generalization across different aspects of antioxidant behavior.

In some cases, the DES structure itself may act synergistically with the functional components, potentially leading to enhanced stability, targeted delivery, or controlled release of active ingredients. This approach of using DES as both a solvent system and functional complex may open new possibilities for formulating and delivering bioactive compounds across various applications in pharmaceuticals, nutraceuticals, and other fields.

The system's artificial intelligence models may be applied to predict and optimize these functional DES formulations, taking into account the complex interactions between components and the resulting physicochemical and biological properties. This may enable the rational design of DES systems tailored for specific functional applications, moving beyond traditional trial-and-error approaches.

Further, this system provides an ability to address the increased pressure on food supply (that is expected to increase) with significant population increases, especially in terms of minorities and low-income communities. The estimates of population growth in most areas indicate that disparities in the educational quality, economic prosperity, and global competitiveness of such minority groups will also grow. To reverse these projections, this system is able to provide new antioxidant combinations, functional DES, and AI system and algorithms aligned with several other initiatives in research not before seen directly focused on the development and application of DES.

Leveraging a blend of advanced data science and experimental food chemistry, this system can develop a reliable and robust method for the rapid evaluation of new antioxidant combinations that can be used as preservatives in broadly used fats and oils that are critical to food security and safety. Beyond the projected developments in terms this system can evaluate synergistic combinations of antioxidants (which are many times more efficient than empirical, hunt-and-peck screening approaches), this system will take advantage of the most advanced methodologies linked to machine learning, opening the door to feed additional data into the predictions, such as weather (temperature, UV, humidity) or traffic forecasts-variables that can influence the degree of oxidation process during storage/transport. This system can be applied to other fields like cosmetics and pharmaceuticals, further increasing the impact of the research.

This system is the development and implementation of an advanced artificial intelligence (AI) system based on molecular fingerprints and chemical descriptors to predict synergistic mixtures of antioxidants, simultaneously accounting for multiple chemical properties of the antioxidants, numerous variables related to the sample, and multiple environmental factors. The system can provide new and more effective combinations of antioxidants.

The system can provide for an existing DES AI model to develop the first series of functional DES, integrating synergistic mixtures of antioxidants in their structure. These stable complexes will retain the antioxidants in proximity and provide the ultimate platform to support synergistic interactions of those antioxidants.

While it can be applied to other industries and applications other than the food industry, this system includes the development of a novel artificial intelligence model with experimental chemistry to develop tactics that will significantly accelerate the discovery of new antioxidant formulations as well as streamline the optimization of existing antioxidant combinations to enhance food safety and maintain food availability and security.

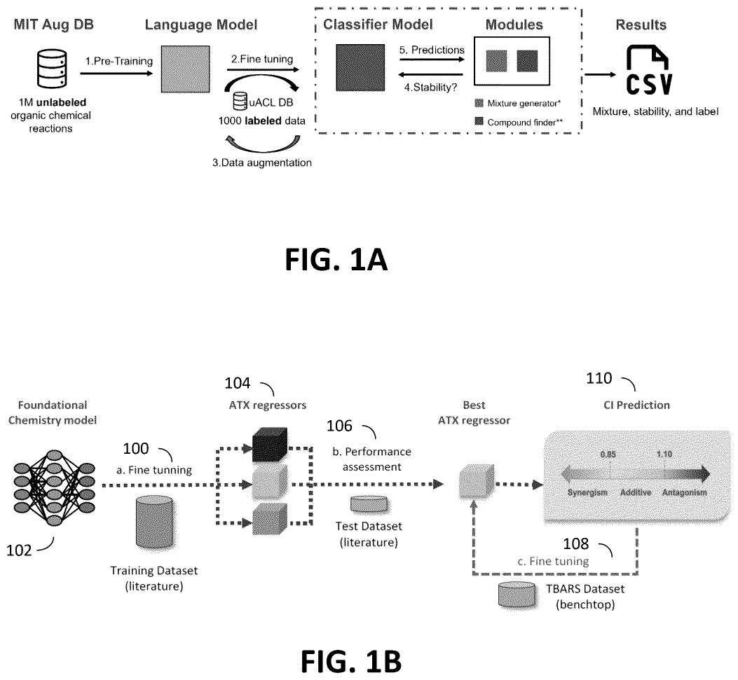

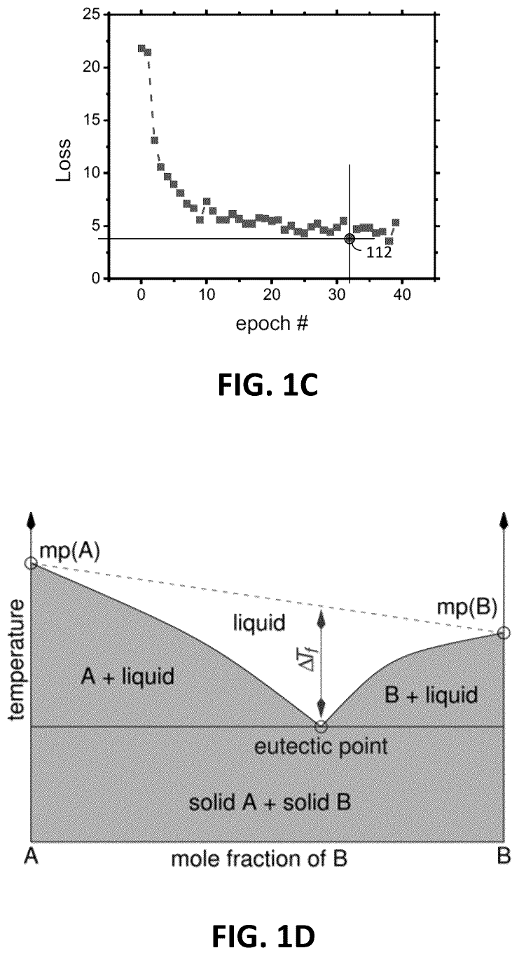

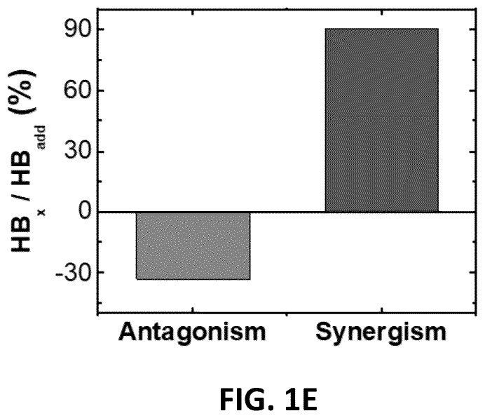

This system pioneers the use of AI to predict the formation of DES via a model that considers hydrogen bonding and that results in a substance with a significantly lower melting point than the individual components (eutectic point, FIG. 1D). Analysis of the system's database, developed to train the model, confirms that the number of hydrogen bonds is also responsible for the type of interactions of the antioxidants. Antioxidant combinations in this database that exhibited a synergistic effect had 90% more hydrogen bonds than those that were additive (FIG. 1E-Percentage of number of hydrogen bonds in synergistic or antagonistic antioxidants) compared to the number of hydrogen bonds in additive mixtures). Conversely, antagonistic combinations displayed 30% less hydrogen bonds than the additive antioxidants.

Referring to FIG. 1A, this system uses an artificial neural network model. An artificial neural network model may be a computational framework inspired by the structure and function of biological neural networks in the brain. This type of model typically consists of interconnected nodes or “neurons” organized in layers. The network may include an input layer that receives data, one or more hidden layers that process the information, and an output layer that produces the final results or predictions. In some implementations, each connection between neurons may be associated with a weight that determines the strength of the signal passed between them. The network may learn to perform tasks by adjusting these weights based on the error between its predictions and the actual outcomes, often through a process called backpropagation. Artificial neural networks may be capable of recognizing complex patterns in data and can be applied to various tasks such as classification, regression, and clustering. In the context of predicting antioxidant interactions, the neural network model may take molecular descriptors and chemical properties as inputs and process this information through its layers to output predictions about potential synergistic effects or other relevant characteristics of antioxidant combinations. The flexibility and learning capabilities of artificial neural networks makes them well-suited for handling the multifaceted nature of antioxidant interactions, potentially accounting for numerous variables and nonlinear relationships that traditional methods might struggle to capture.

The artificial neural network model can be pre-trained using general unlabeled chemical data and then fined-tuned as a binary classifier using an ad-hoc database (uACL DB) containing 1200 examples of DES/NADES from the literature. Within these, 800 mixtures are examples of stable DES/NADES (labeled as 1), and 200 entries contain examples of mixtures that either do not form DES/NADES or that are not stable (labeled as 0). While this asymmetry reflects what is typically published (primarily positive results), the imbalance will likely lead to overoptimistic predictions. To overcome this issue, we considered the (extremely low) probability of generating stable DES/NADES by mixing random chemicals at random stoichiometric coefficients. Thus, the database was augmented by generating one million random mixtures, which were labeled as zero (unstable). Upon optimization, our model was able to calculate the probability of formation for millions of combinations in just a few minutes, significantly facilitating the discovery process. These results, which were experimentally validated by generating stable DES incorporating pharmaceutical compounds, demonstrated the model's capacity to identify the intricate interplay of variables involved in the formation of hydrogen bonding.

In one embodiment, the system allows for the understanding that the intricate interplay of variables controlling hydrogen bonding is critical for a wide number of scientific fields, and the central theme of this proposal. Toward that goal, this system use of AI methods (e.g., language-based models) to predict the formation of hydrogen bonds in various systems, identifying new synergistic mixtures of antioxidants and predicting the formation of novel DES. The system can provide significantly better predictive capabilities but also enable the application of existing knowledge related to hydrogen bonding towards a more rational and efficient use of antioxidants for food applications. This system brings a combination of innovative strategies in data science and experimental food chemistry to bear on a significant problem—antioxidant development—that is directly relevant to food and nutrition safety and security.

This system blends the development of novel artificial intelligence models with experimental chemistry to develop tactics that will significantly accelerate the discovery of new antioxidant formulations as well as streamline the optimization of existing antioxidant combinations to enhance food safety and maintain food availability and security. This system addresses the problem with the current technology and gap in knowledge by using a learning model and an artificial intelligence model based on deep learning architecture to both predict the type of interaction (synergistic, additive, and antagonistic) of known mixtures as well as to unveil new antioxidant combinations. Each mixture can be associated with a combination index value (CI) or other available metric and used as input for our model, which was challenged against a test dataset containing experimental results generated for that purpose.

In one embodiment, the system is based on the use of Simplified Molecular Input Line Entry System (SMILES) notation to represent the antioxidants combinations as text representations. Each mixture is then associated to a combination index value (CI), an established metric often used to assess the magnitude of these interactions. The system also utilizes a self-data augmentation method to overcome overfitting due to the limited amount of data for the training step. This strategy was implemented by representing the stoichiometric ratio as a repetition of the same antioxidant compound instead of numerical representations (see FIG. 1B), allowing the rearrangement of the SMILES strings to all possible non-repeated positions in the final mixture. In this sense, the use of chemical descriptors (density, functional groups, polarity, etc.) can be avoided, reducing the complexity of the AI model, and easily allowing its implementation in benchmark routines. The performance capability of the model (e.g., computer program that is designed to simulate what might or what did happen in a situation) was first assessed by predicting CI values using a database developed from literature reports (n=700), showing a relatively good agreement (R²ₜₑₛₜ=0.92 and R²ₜᵣₐᵢₙ=0.95) between the predicted output and the actual value for both the training (n=560) and test (n=140) datasets.

This AI model was enhanced with various amounts of experimental data (antioxidant power data assessed by the TBARS assay) collected using lard samples, which were used as a non-exclusive example to demonstrate the capabilities of the system. This approach allowed the model to learn from the experimental chemical space that was not specifically described in the surveyed literature. The results show that significant improvements in the model's performance were obtained as the amount of fine-tuning data increased, increasing the correlation between the predicted and experimental results from R²=0.01 (poor correlation) to an R² value of 0.90 (improved correlation). These results not only demonstrate the predictive power of the proposed algorithm but also the importance of having chemically relevant experimental data to enhance the model's performance and provide suitable predictions with statistical relevance.

The predictive model may be enhanced through various approaches that include incorporation of experimental data: The model's performance may be improved by integrating chemically relevant experimental data, such as antioxidant power measurements from TBARS assays. This approach may allow the model to learn from real-world chemical interactions not fully captured in literature-based datasets. Fine-tuning with diverse samples can be used that includes where the model may be enhanced by fine-tuning it with data from various food matrices beyond lard samples. This may include oils, meats, or plant-based products, potentially expanding the model's applicability across different food systems. The enhancement can include environmental factor integration where the predictive capabilities may be augmented by incorporating environmental variables such as temperature, humidity, and UV exposure. This may enable the model to account for storage and transport conditions that influence oxidation processes. Temporal data analysis can be used so that the model may be improved by analyzing time-series data of antioxidant effectiveness. This approach may allow for predictions of how antioxidant combinations perform over extended storage periods. Molecular descriptor expansion can be used so that the model's predictive power may be enhanced by incorporating additional molecular descriptors beyond those initially used. This may include parameters related to molecular size, polarity, or electronic properties of antioxidants. Cross-validation techniques: can be Implemented to provide for cross-validation methods used to improve the model's generalizability and reduce overfitting, potentially leading to more reliable predictions across diverse antioxidant combinations. Ensemble learning approaches can be used that can combining multiple models or algorithms may enhance overall predictive performance by leveraging the strengths of different machine learning techniques. Adaptive learning implementation can be used so that the model may be designed to continuously learn and update its predictions based on new experimental results, potentially improving its accuracy over time as more data becomes available.

In one embodiment the model and its algorithms were initially trained using the MIT Mixed Augmented database to generate a foundational general chemistry model. Then, this model was improved by the following: first, data splitting and augmentation; second, model fine-tuning and testing, and third by fine-tuning with chemically-relevant experimental data. First, the original database (e.g., one that is developed in house from literature reports) was randomly divided into a training dataset (80% of the database) and a test dataset (20% of the database). For both cases, the stoichiometric ratio of the mixture of antioxidants was represented either by repetitions of the same antioxidant in the SMILES notations or by numbers, as described in the experimental section of this manuscript. Second, both versions of the training dataset (numerical or textual) were used to fine-tune the foundational general chemistry model into a unique AI regressor to predict CI values or another pertinent metric. Then, the performance of all the generated antioxidant regressors was assessed by using the corresponding test dataset (textual or numerical) to measure key metrics such as root-mean-square error (RMSE), mean absolute percentage error (MAPE), as well as R². Finally, the regressor with the best predictive capability was enhanced by incorporating different amounts of benchtop data (e.g., antioxidant capacity of binary mixtures of phenolic antioxidant).

An overview of these steps is represented in FIG. 1B which is an overview of the process to fine-tune at 100 the foundational general chemistry model 102 into several antioxidant regressors 104. These are followed by their respective performance assessments 106. The best antioxidant regressor was then fine-tuned with benchtop data 108 to enhance the CI prediction capability 110 of the model with respect to mixtures of phenolic antioxidants. The relationship between CI values and antioxidant behavior is described below.

In addressing problems with the current state of the art, this system fine-tunes a general foundational chemistry model into a regressor, with the ability to predict the behavior (antagonistic, additive, or synergistic) of antioxidant mixtures. The general chemistry model was pre-trained by using the well-known USPTO-MIT mixed augmented database that contains approximately one million unlabeled organic chemical reactions. Briefly, this step was included to increase the model's vocabulary (˜5000 unique tokens) by providing sufficient chemical information in the form of text notation. Unlabeled data may include a wide range of information about chemical compounds, reactions, and properties without predefined classifications or target variables. This type of data can encompass molecular structures, chemical formulas, reaction conditions, physical properties, and spectroscopic measurements. In some cases, unlabeled chemical data may be represented using standardized formats such as SMILES notation, InChl keys, or molecular fingerprints.

The data may be derived from various sources including scientific literature, experimental results, chemical databases, and computational simulations. Unlabeled chemical data can provide a rich foundation for machine learning models to extract patterns and relationships without being constrained by predetermined categories. This approach may allow the system to discover novel insights or unexpected correlations within the chemical space. By leveraging large volumes of unlabeled chemical data, models can potentially develop a more comprehensive understanding of chemical behavior and interactions, which may be particularly valuable for tasks such as predicting antioxidant synergism or identifying new functional materials.

Moreover, the parameters such as weights and bias can be continuously adjusted during the training session (training dataset) to improve the model's performance using unseen chemical data (test dataset). This task was accomplished by monitoring the output of the loss function (e.g. “the loss”) versus the number of epochs, leading to a loss of 4.00 at epoch number 32 (FIG. 1C), which was considered acceptable. On the other hand, the loss for the training dataset at the same epoch number was 3.87, suggesting that a convergence point was reached by using both datasets and thus suggesting that more training was unlikely to further improve the model. The generated foundational chemistry model was then fine-tuned into several antioxidant regressors under different data representation scenarios.

In some aspects, the fine-tuning process may involve adjusting the pre-trained foundational chemistry model to specialize in predicting antioxidant interactions. This process may utilize a smaller dataset of labeled antioxidant combinations and their known interaction types (synergistic, additive, or antagonistic). The fine-tuning step may involve freezing some of the earlier layers of the neural network while allowing the later layers to be updated. This approach may help retain the general chemical knowledge learned from the larger unlabeled dataset while adapting the model's output layers to the specific task of predicting antioxidant behavior.

During fine-tuning, the model may be exposed to antioxidant-specific data, potentially including SMILES notations of antioxidant compounds, their combination ratios, and corresponding combination index (CI) values. The model's parameters may be incrementally adjusted using techniques such as backpropagation and gradient descent to minimize the difference between predicted and actual CI values. In some implementations, the fine-tuning process may incorporate a technique called transfer learning, where knowledge gained from the general chemistry domain is transferred and adapted to the specific domain of antioxidant interactions. This approach may allow the model to leverage its broad understanding of chemical principles while developing specialized predictive capabilities for antioxidant synergism.

The fine-tuning step may also involve adjusting hyperparameters such as learning rate, batch size, and number of epochs to optimize the model's performance on the antioxidant prediction task. Cross-validation techniques may be employed to ensure the model generalizes well to unseen antioxidant combinations. In some cases, the fine-tuning process may be iterative, with multiple rounds of adjustment and evaluation using different subsets of the antioxidant dataset. This iterative approach may help refine the model's predictive accuracy and robustness across various types of antioxidant combinations.

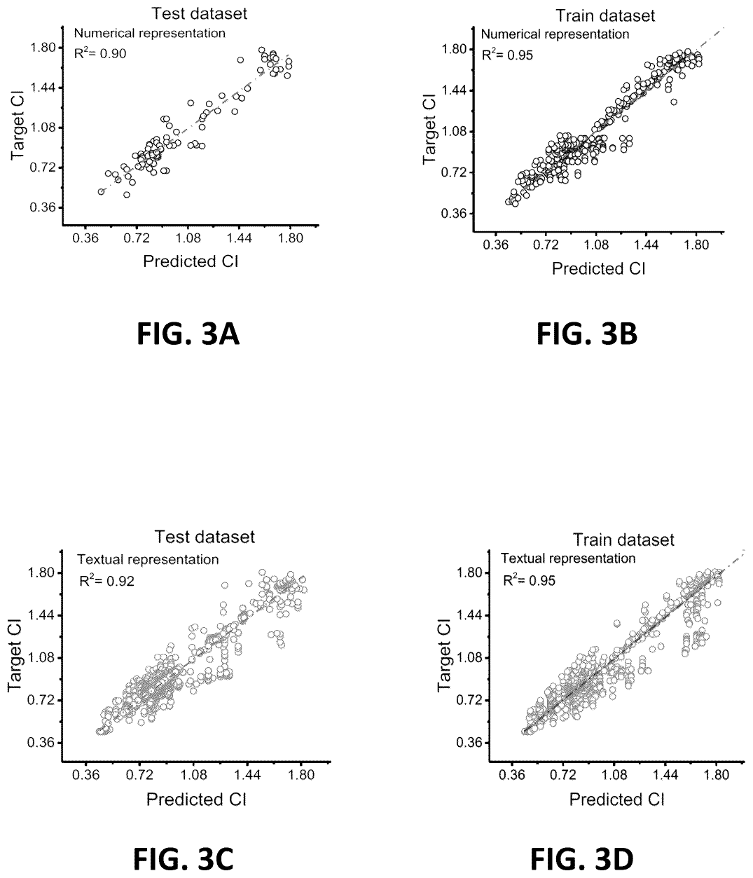

A database (referred to as ATX_uACL db) was developed in-house from previous literature reports and used to fine-tune the last layer of the foundational general chemistry into several regressors. The database contains approximately 1100 combinations (binary and tertiary) in the SMILES notation along with quantitative metrics regarding their antioxidant power such as combination index (CI), the difference in FRAP, % of the synergistic or antagonistic effect as well as Trolox equivalent antioxidant capacity (TEAC). Among those, mixtures with their respective combination indexes are the most common entry in our database (approximately 700) and, given their abundance, selected to fine-tune the general chemistry model into the regressors. For comparison purposes, our database displays 297 entries describing synergistic or antagonistic effects in terms of percentage, 161 for TEAC, and 85 for the differences in FRAP. Therefore, the use of CI was considered most appropriate in one embodiment for the proposed task since a higher number of antioxidant combinations leads to a more representative chemical space and to a more robust and accurate regressor. Aiming to further increase the total number of antioxidant mixtures, the stoichiometric number of each combination was represented as repetitions of the SMILES strings rather than the numerical value itself (vide infra, FIG. 3). In this sense, a mixture that contains two components (A and B) in the molar ratio 2:3 would render 10 unique combinations (permutations of B A B A B, for example). The proposed strategy was then implemented and compared to the use of numerical representation for the molar ratio (e.g., 2A 3B) during the model's fine-tuning into regressors as summarized in Table 1 which summarized results for fine-tuning the general chemistry model into regressor with numerical and textual representations. (1) RMSE: root mean square deviation; (2) MAPE: mean absolute percentage error.

$$RMSE = \sqrt{{\sum }_{i = 1}^{n} \frac{{( {\hat{y}}_{i} - {y}_{i} )}^{2}}{n}} \qquad ( 1 )$$

where ŷᵢ is predicted and yᵢ is the true values.

$$M = \frac{1}{n} {\sum }_{t = 1}^{n} \vert \frac{{A}_{t} - {F}_{t}}{{A}_{t}} \vert \qquad ( 2 )$$

where Aₜ is the true value and Fₜ is the predicted value.

TABLE 1

| | | Test dataset | | Train dataset | |
|---|---|---|---|---|---|
| | | | MAPE | | MAPE |
| Model | Epoch | RMSE | (%) | RMSE | (%) |
| Numerical | 001 | 3.77 × 10⁻² | 17.5 | 1.33 c 10⁻¹ | 20.0 |
| | 005 | 4.59 × 10⁻² | 16.2 | 1.00 × 10⁻¹ | 17.0 |
| | 010 | 2.90 × 10⁻² | 12.9 | 6.20 × 10⁻² | 12.3 |
| | 025 | 1.82 × 10⁻² | 10.1 | 3.90 × 10⁻² | 12.0 |
| | 100 | 1.21 × 10⁻² | 8.73 | 2.10 × 10⁻² | 7.34 |
| | 250 | 1.24 × 10⁻² | 8.65 | 6.30 × 10⁻³ | 5.50 |
| | 350 | 1.07 × 10⁻² | 7.76 | 1.20 × 10⁻² | 5.24 |
| | 500 | 1.19 × 10⁻² | 8.11 | 1.10 × 10⁻² | 4.97 |
| | 750 | 1.37 × 10⁻² | 9.21 | 1.00 × 10⁻² | 4.82 |
| Textual | 001 | 3.90 × 10⁻² | 13.9 | 6.10 × 10⁻² | 16.4 |
| | 005 | 2.90 × 10⁻² | 8.81 | 3.00 × 10⁻² | 11.5 |
| | 010 | 2.73 × 10⁻² | 11.3 | 2.90 × 10⁻² | 5.60 |
| | 025 | 1.95 × 10⁻² | 9.49 | 2.50 × 10⁻² | 5.27 |
| | 100 | 1.24 × 10⁻² | 7.94 | 9.70 × 10⁻³ | 6.21 |
| | 250 | 1.13 × 10⁻² | 7.02 | 9.30 × 10⁻³ | 4.92 |
| | 350 | 1.04 × 10⁻² | 7.01 | 8.60 × 10⁻³ | 4.60 |
| | 500 | 1.01 × 10⁻² | 6.64 | 5.26 × 10⁻³ | 3.72 |
| | 750 | 1.17 × 10⁻² | 6.97 | 5.10 × 10⁻³ | 3.76 |

Regarding the models fine-tuned with numerical representations, it can be observed that a minimum value of root means square deviation (RMSE=1.07×10⁻²) and mean absolute percentage error (MAPE=7.76) was achieved at epoch 350 (˜20 minutes) for the test dataset. In other words, the combination index (CI) predicted by the model differs (on average) by 7.76% from the ground truth value. Any additional increase in training negatively impacted the performance of the algorithm assessing new data, indicating that the neural network was overfit. This idea was also supported by the fact that the RMSE and MAPE are continuously decreasing while assessing the training dataset. On the other hand, the models fine-tuned with text representations performed better (RMSE=1.01×10⁻² and MAPE=6.64) at epoch 500, suggesting that the textual model requires more training (˜30 minutes) to achieve its best performance. To further show the prediction capability of both model types at their respective best epoch, the predicted CI was evaluated versus the target CI, results that are summarized in FIGS. 3A through 3D.

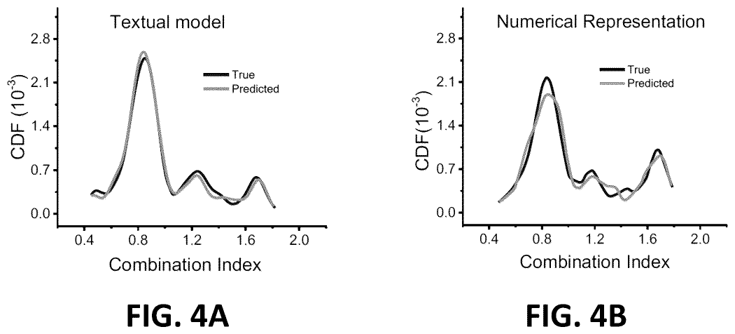

The textual and numerical models displayed a good agreement (R²=0.92 and R²=0.90, respectively) with respect to the target CI in the test dataset, demonstrating a satisfactory prediction capability at their best epochs. For both models, a R² of 0.95 was achieved assessing the train dataset (FIGS. 3B and 3D), which indicates that the neural network (for the textual model) is slightly better at predicting unseen data since its test R 2 (0.92) is closer to the optimal one (0.95). Although only a slight difference between the two models was observed, the use of textual representations could benefit the neural network's performance in cases where a limited amount of training data is available. In this scenario, a few experimental points (or data from the literature) could be augmented and then used to train the algorithm. For demonstration purposes, decreasing the size of the training dataset by half still allowed the textual model to display an acceptable performance (R²=0.86); while cutting the numerical set by half rendered significantly poorer performances (R²=0.61) when assessing the antioxidant combinations present in the test dataset (data not shown). Aiming to get further insights regarding the performance of both models, the cumulative distribution function (CDF) was plotted versus the combination index for the true and predicted values (test dataset), as shown in FIGS. 4A and 4B.

The cumulative distribution of the true combination index (blue line-lower peak FIG. 4A, higher peak FIG. 4B) falls in three main regions, displaying a higher CDF at a CI value around 0.85. In other words, there is a higher probability of finding combination indexes with values equal to or lower than 0.85 (synergistic interactions) in the test datasets, a finding that is aligned with the structure of the existing dataset. This characteristic was also observed in the training dataset, indicating that the data splitting of the original database into the two subsets (train and test) was unbiased and representative. Moreover, there is a good agreement between the CDF for the predicted values for the textual model (FIG. 4A, orange line) and the true output (FIG. 4A, blue line) in all three regions. On other hand, the predicted CDF for the numerical model (FIG. 4B, orange line) is slightly off in all three regions, especially the one with CI>1.10. Therefore, the textual model was selected and then applied to predict the behavior of antioxidant mixtures.

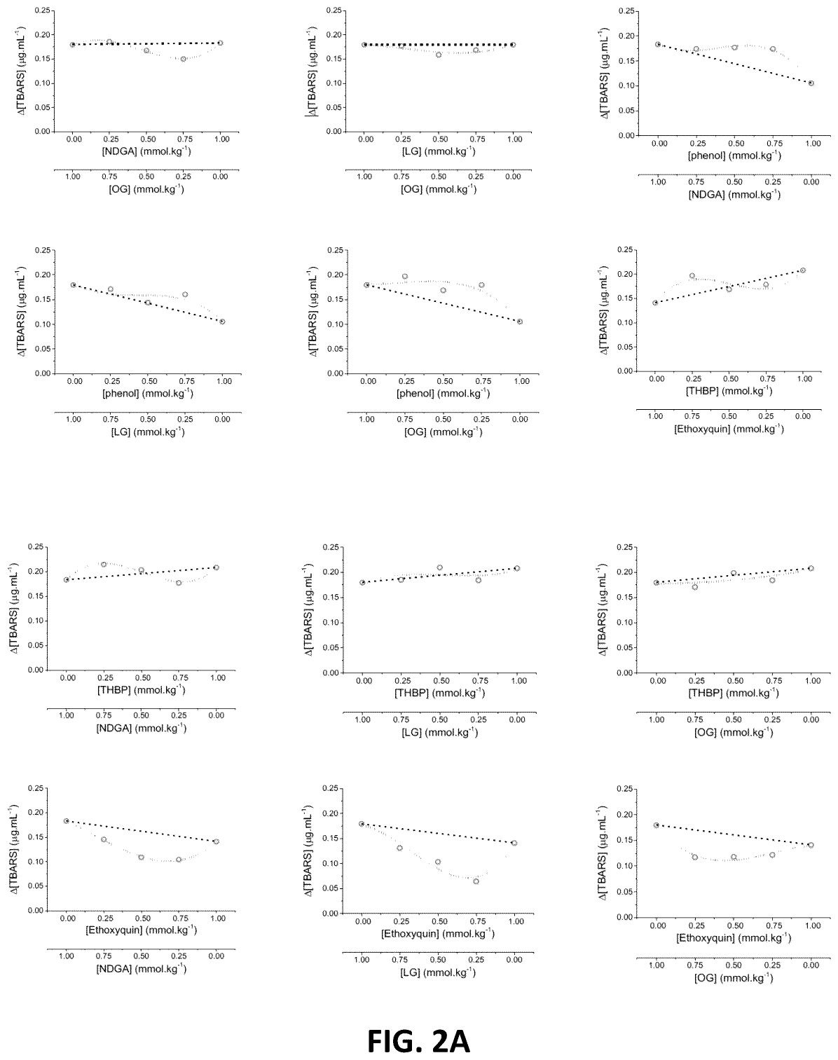

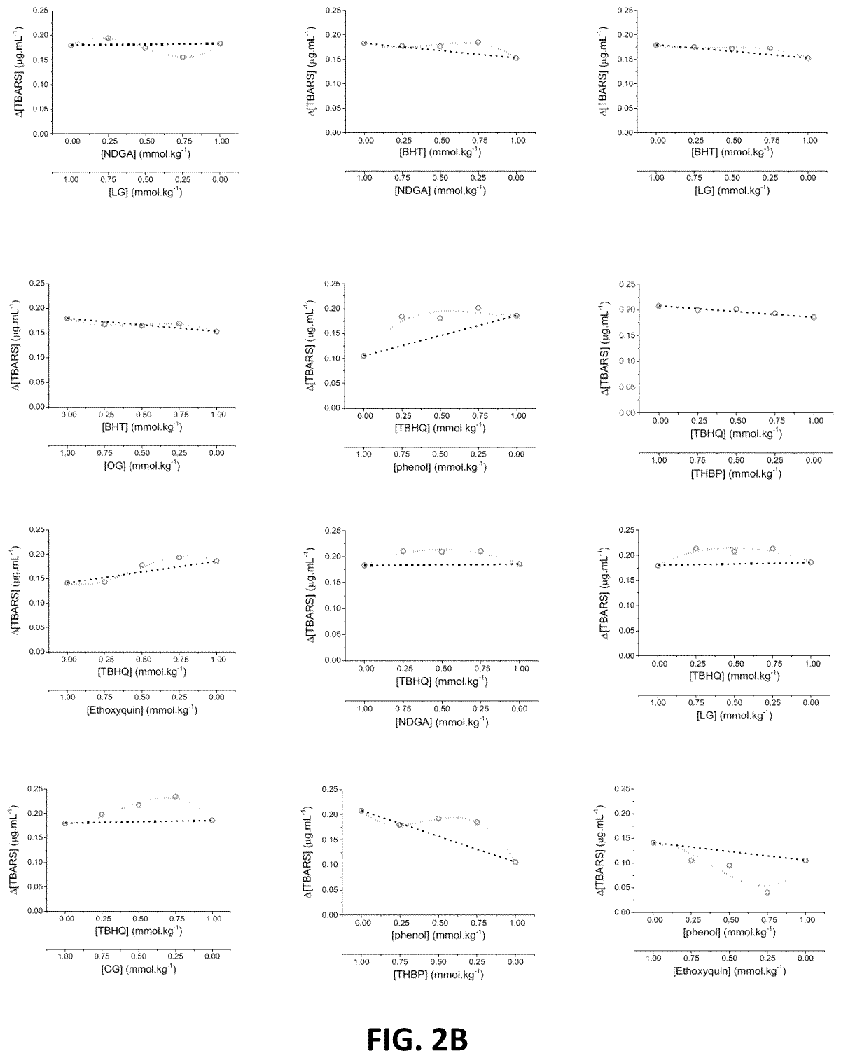

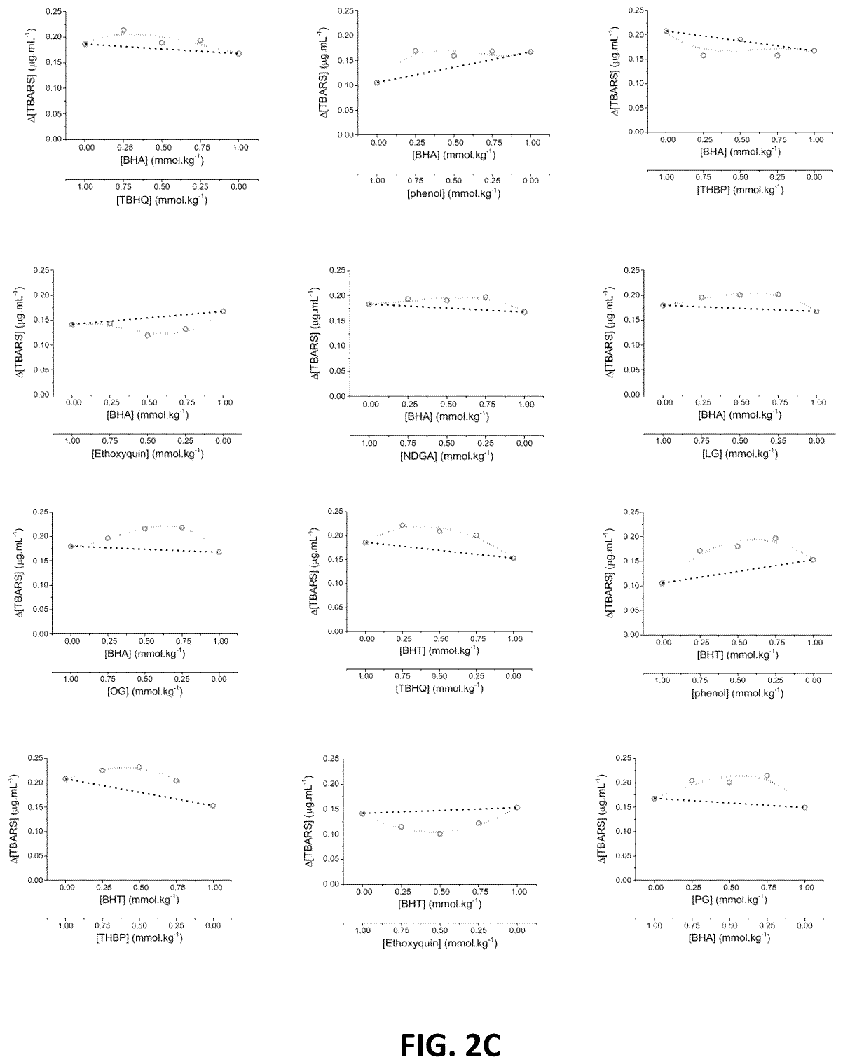

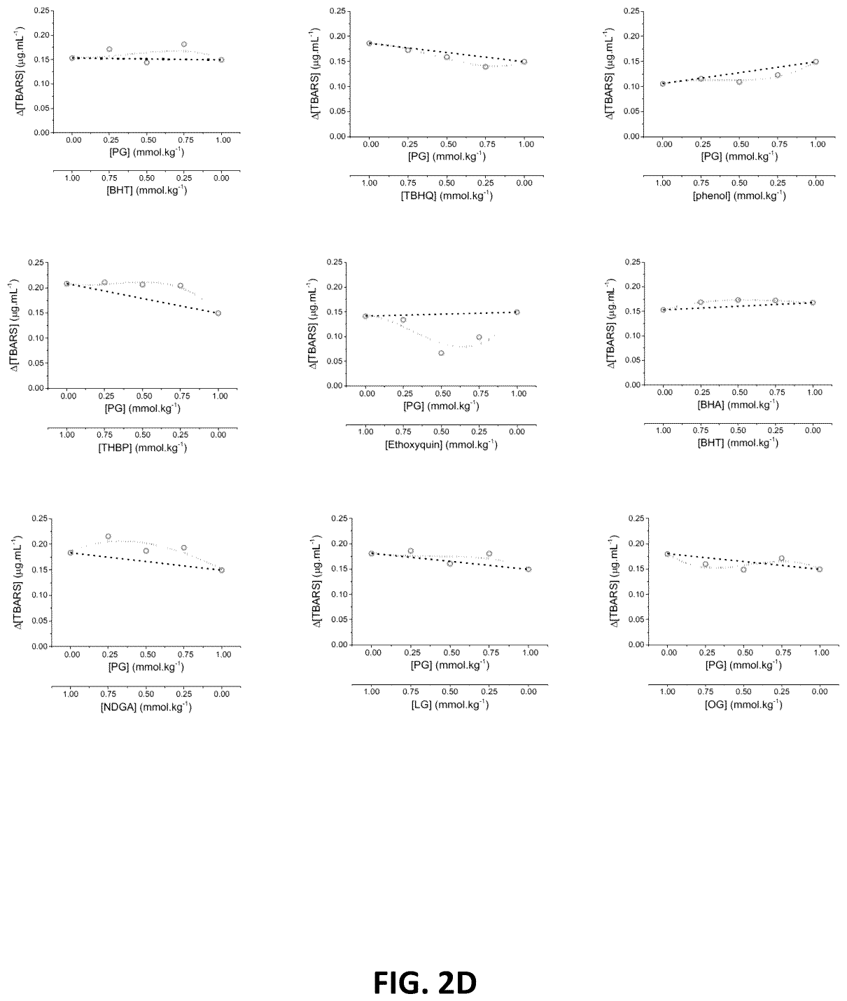

The antioxidant power of binary combinations of common compounds was investigated by using an accepted methodology, in this case the thiobarbituric acid reactive substances (TBARS) assay and lard as a lipidic substrate. Briefly, thiobarbituric acid undergoes a complex reaction with malondialdehyde (MDA), a well-known marker for oxidative stress in samples containing lipids, rendering a pink chromogen compound that can be quantitatively assessed via spectrophotometry. Thus, the magnitude of oxidative stress can be easily related to absorbance changes, allowing for an assessment of the rancidity in food samples in the presence or absence of antioxidants. In this scenario, lard samples were prepared with binary combinations of 10 phenolic antioxidants, incubated in a convection oven, and the resulting absorbance was compared to the absorbance generated in samples containing the individual components. The data analysis was accomplished through the use of a graphical method similar to an isobologram (see FIGS. 2A to 2D) allowing for the calculation of the difference (ΔTBARS) between the absorbance obtained with samples containing antioxidants and the individual controls. Thus, synergistic combinations would render ΔTBARS>0, while antagonistic interactions would generate ΔTBARS<0. Following the same rationale, mixtures leading to ΔTBARS values that showed only small differences with respect to the baseline, were considered to feature an additive antioxidant behavior. Also, in line with this analysis, ΔTBARS values will be inversely proportional to the CI value selected, among other possible metrics, as outcome for the model. Thus, a higher degree of synergistic antioxidant interactions results in a lower CI and in an increase in ΔTBARS.

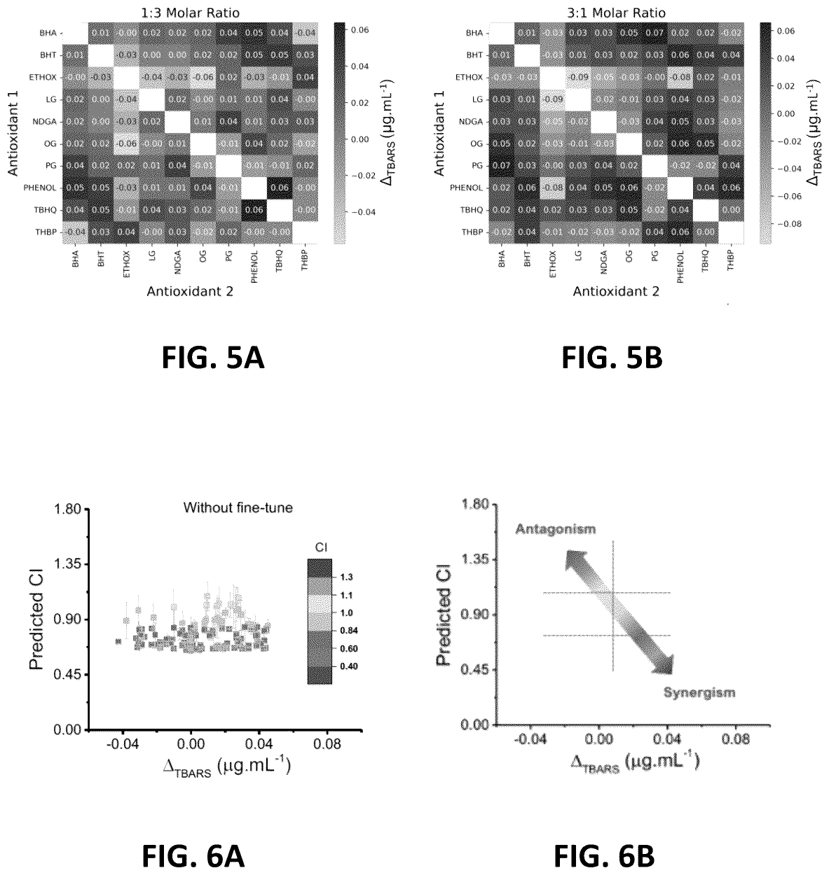

It is interesting to note that mixtures containing ETHOX mostly rendered antagonistic interactions (light pink shade, FIGS. 5A and 5B). On other hand, the vast majority of the synergistic interactions (dark purple shade, FIGS. 5A and 5B) included mixtures containing either BHA or BHT, a finding that is also in good agreement with known information. It is also important to note that ΔTBARS values in the +0.1 were obtained, defining reasonable limits for our model, and allowing us to focus on mixtures featuring −0.05<ΔTBARS>+0.06.

Results provided strong evidence about the predictive power of the regressor trained and tested with textual representations of ˜1100 antioxidants mixtures form the literature (R²=0.92). Therefore, it was reasonable to expect that our approach would provide accurate predictions of the CI values of the binary mixtures of phenolic antioxidant (see Table 2 below). Towards this goal, the antioxidant combinations used to obtain the data shown in FIGS. 5A and 5B were translated into SMILES format and used as input for the textual model with the best performance. The predicted combination index for those mixtures versus its ground normalized experimental value (ΔTBARS), is presented in FIG. 6A. It is important to state that ΔTBARS values (proportional to the degree of oxidation) are inversely proportional to the CI value (but possibly related to other metrics such as antioxidant power), which was selected as the outcome for the model in one embodiment. Thus, a higher degree of synergistic antioxidant interactions results in a lower CI and in an increase in ΔTBARS, as shown in FIG. 6B. Therefore, in one embodiment and as a last step, the antioxidant combinations used to obtain the experimental data were translated into SMILES format, and used as input for the best regressor, to calculate CI values. While this analysis was expected to lead to a simple correlation (as shown in FIG. 6B), the preliminary data analysis (presented in FIG. 6A) showed no evident correlation between the CI value predicted by the model and the ΔTBARS values experimentally obtained.

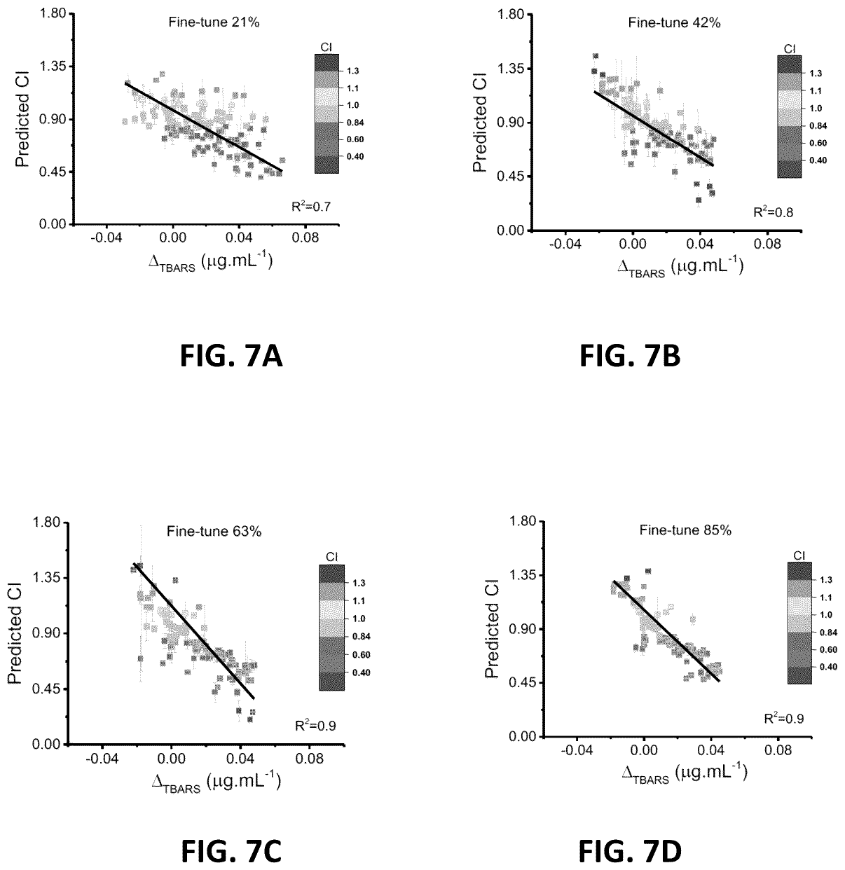

Therefore, second fine-tuning step was implemented where increasing amounts of experimental data (ΔTBARS as a function of CI) were presented to the model during the second fine-tuning step (FIG. 1B, 108), where increasing amounts of experimental data (SMILES notation and ΔTBARS represented in terms of CI) were presented to the model. The predicted CI value (for multiple mixtures of antioxidants) was evaluated as a function of the experimental ΔTBARS. A fine-tuning step with just 21% of the experimental data (e.g., 47 randomly selected data points) allowed the model to capture enough chemical patterns to render a positive correlation between the two variables (using the test dataset) as more experimental data was presented, the model was able to identify the chemical patterns within each group, leading to more accurate predictions of the CI values. When the model was fine-tuned with 85% of the experimental data (191 randomly selected datapoints), a significantly better agreement was obtained. These results not only support the possibility of using this model for predicting the behavior of the selected antioxidant mixtures but also highlight the importance of obtaining relevant benchtop data to support the computational approach. A summary of the results of the analysis are shown in FIGS. 7A through 7D, where the correspondence between the two parameters was evaluated as a function of the amount (% of the experimental dataset, ˜225 entries) used for the fine-tuning step. As shown in FIG. 7D, this process renders an acceptable agreement and lower dispersion of the data, indicating the possibility of using this model for predicting the behavior of the selected antioxidant mixtures.

Using Artificial Intelligence based on deep learning architecture to predict antioxidant interactions (synergism, additive, and antagonism) by using SMILES notation and a combination index factor as input parameters for the model improves on existing technology. The best generated algorithm (R²ₜₑₛₜ=0.92 and R²ₜᵣₐᵢₙ=0.95; assessing our proprietary database) was achieved through a new data augmentation strategy where the stoichiometric number was replaced by repetitions of its respective antioxidant compound in the SMILES format. This approach leads to a more representative chemical space during the model training, which addresses common overfitting problems due to the use of relatively small datasets. Then, the predictive capability of the algorithm was challenged against experimental benchtop data collected through TBARS assay. As a result, an expected inverse correlation between the predicted CI and ΔTBARS increases (R²=0.7 to 0.9) as the amount of fine-tuning data increases (21% to 85%), suggesting that the model successfully recognized chemical patterns from the antioxidant compounds used in the experimental analysis. We believe that the proposed method could be used as an auxiliary tool in benchmark analysis routines, offering a novel strategy to enable broader and more rational predictions related to the antioxidant mixtures behavior.

In one embodiment and one testing scenario, the computational work presented in this manuscript was carried out in the Palmetto Cluster at Clemson University. The node was set to 32 cores (ncₚᵤₛ) and the allocated memory was set to 372 Gb. As a graphical processing unit (GPU), a NVIDIA Tesla V100 was used to train the foundational chemistry model as well as to fine-tune the generated model into the regressors. It is important to state that while access to the cluster was critical to speed up the initial training of the foundational chemistry model, the trained algorithm can be executed in a standard computer.

The proprietary antioxidant database (referred to as uACL-DB) was developed by manually retrieving data from the literature, and includes various antioxidant molecules, solvents, samples, and experimental conditions for their evaluation. In all cases, the antioxidant combinations were represented in SMILES notation along with their molar ratio and respective metric to measure the degree of interaction such as combination index (CI), the difference in FRAP, % of the synergistic or antagonistic effect as well as Trolox equivalent antioxidant capacity (TEAC). The resulting database displayed approximately 1100 entries, and the gathered data was analyzed by a Python algorithm to avoid duplicates.

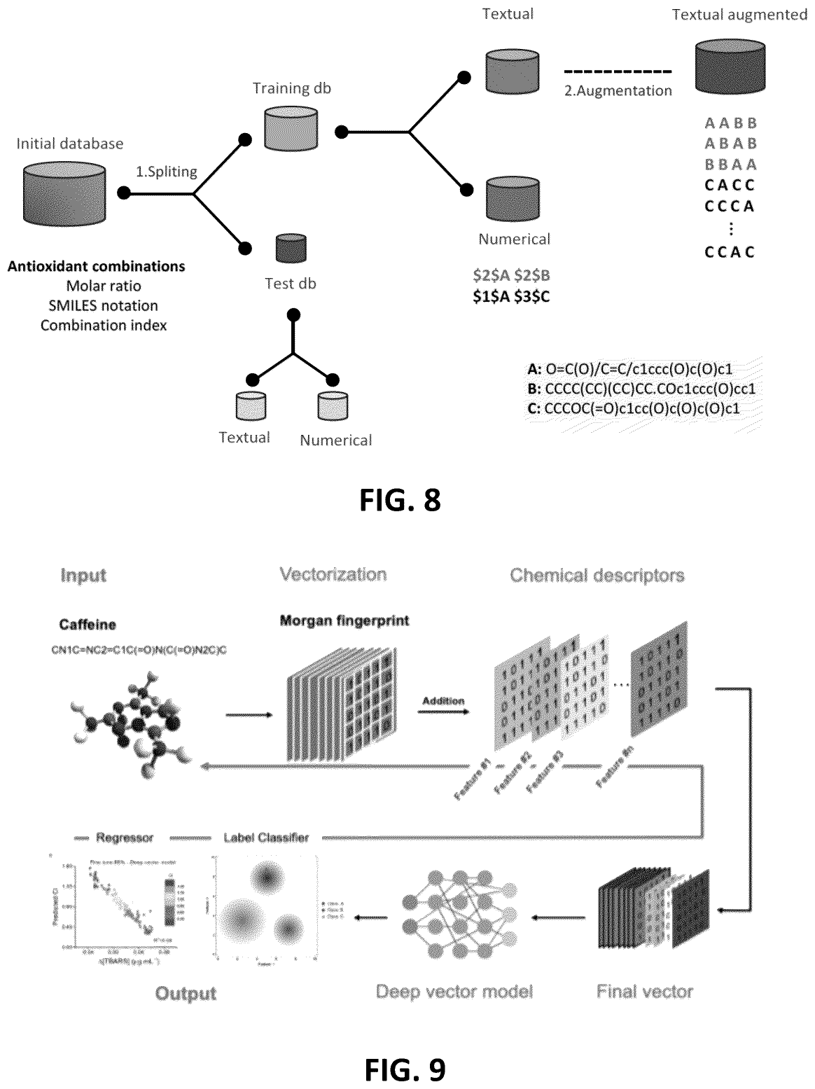

The uACL-DB was randomly split into training (85%) and test dataset (15%). Aiming to avoid data leaking between the datasets, all the files were compared by an algorithm in addition to an extra manual check of each entry. Then, each dataset was duplicated and assigned as a textual or numerical representation, giving a total of 4 databases (numerical train, numerical test, textual train, textual test). For the pair assigned as numerical, the stoichiometric ratio of all combinations was represented as a number in between a non-SMILES special character (e.g., $3$A $1$B). On other hand, for the pair assigned as textual, the stoichiometric ratio was represented as repetitions of the antioxidants (e.g., A A A B). Then, a python algorithm was used to augment the textual datasets by permuting the antioxidant smiles to all possible non-repeated positions in the final mixture. These processes are presented in FIG. 8.

The foundational general chemistry model was developed by transformer-type architectures used in Natural Language Processing and are pre-trained using Self-Supervised Learning on large amounts of text data to create Foundation Models for different human languages. This pre-training gives the model a broad understanding of the language(s), allowing it to quickly and efficiently adapt to specific downstream tasks. A general chemistry model was pre-trained on a large corpus of chemical reaction information, which was then fine-tuned for a specific task. The ELECTRA deep learning model used in this manuscript had 4 hidden layers for the generator and 16 for the discriminator, with a vocabulary size of 30,000 and 40 training epochs. The output model containing all trained parameters was stored in a directory called foundational general chemistry model. The molecular Transformer USPTO-MIT Mixed Augmented database was used to train as well as to evaluate the proposed chemistry model.

Both the numerical and the textual database were used to fine-tune the last layer of general chemistry foundational model into a regressor. The appropriate test dataset for each stoichiometric representation type (text or numerical) was used to investigate the performance of the generated regressor by assessing unseen antioxidant mixtures by the algorithm. Regarding the neural network architecture, the parameters “max seq length”, “train batch size”, and “learning rate” were adjusted to 128, 32, and 4E-5, respectively.

The oxidative process of a commercial organic pork lard (Fatworks, Premium Cooking Oil, EST.M-8757) under heating conditions (85° C. for 4 h) was evaluated after adding the corresponding antioxidants. The antioxidants were either individual components or binary mixtures of 10 different phenolic antioxidants, at different ratios. Standard solutions of each antioxidant (propyl gallate, PG, 2,4,5-trihydroxybutirophenone, TBHP, tert-butylhydroquinone, TBHQ, nordihydroguaiaretic acid, NDGA, tert-butyl-4-hydroxyanisole, BHA, 2,6 di-tert-butyl-4hidroxymethylphenol, Phenol, 3,5 di-tert-butyl-4-hydroxytoluene, BHT, lauryl gallate, LG, octyl gallate, OG, ethoxyquin, ETHO) were obtained by dissolving a known amount of the pure standard in ethanol. The abbreviation of each antioxidant compound can be found in. Then, each antioxidant was incorporated in different aliquots of the commercial lard. Accordingly, 15 g of pre-melted commercial lard were mixed with 200 μL of the antioxidant standard solution to acquire a final concentration of 1 mmol·kg⁻¹ of antioxidant. To prepare 1 mL of the antioxidant's binary combinations, proper volumes of lard containing the antioxidants were combined to get antioxidant ratios of 1/4:3/4, 2/4:2/4, and 3/4:1/4, respectively. Under those selected experimental conditions, 145 samples of lard containing antioxidants were evaluated. The oxidative effect of prepared lard samples was evaluated by the TBARS assay. The analytical procedure was carried out by adding 100 μL of lard to a glass vial containing 2 mL reagent solution (Thiobarbituric acid/trichloroacetic acid/hydrochloric acid mixture). Then, the reaction mix was heated up in a hot bath set at 100° C. for 15 min. Consequently, the colorless starting solution turned to a pink colored solution which developed an absorption band centered at 533 nm. Before spectrophotometric measurements, the resulting pink solution was centrifuged at 14500 rpm for 5 min.

TABLE 2

| | Molar | |
|---|---|---|
| Combination | Ratio | Canonical SMILES |
| PG BHA | 1:3 | CCCOC(═O)c1cc(O)c(O)c(O)c1 |
| | | CCCC(CC)(CC)CC.COc1ccc(O)cc1 |
| | | CCCC(CC)(CC)CC.COc1ccc(O)cc1 |
| | | CCCC(CC)(CC)CC.COc1ccc(O)cc1 |
| PG BHA | 1:1 | CCCOC(═O)c1cc(O)c(O)c(O)c1 |
| | | CCCC(CC)(CC)CC.COc1ccc(O)cc1 |
| PG BHA | 3:1 | CCCOC(═O)c1cc(O)c(O)c(O)c1 |
| | | CCCOC(═O)c1cc(O)c(O)c(O)c1 |
| | | CCCOC(═O)c1cc(O)c(O)c(O)c1 |
| | | CCCC(CC)(CC)CC.COc1ccc(O)cc1 |
| PG BHT | 1:3 | CCCOC(═O)c1cc(O)c(O)c(O)c1 |
| | | Cc1cc(C(C)(C)C)c(O)c(C(C)(C)C)c1 |
| | | Cc1cc(C(C)(C)C)c(O)c(C(C)(C)C)c1 |
| | | Cc1cc(C(C)(C)C)c(O)c(C(C)(C)C)c1 |
| PG BHT | 1:1 | CCCOC(═O)c1cc(O)c(O)c(O)c1 |
| | | Cc1cc(C(C)(C)C)c(O)c(C(C)(C)C)c1 |
| PG BHT | 3:1 | CCCOC(═O)c1cc(O)c(O)c(O)c1 |
| | | CCCOC(═O)c1cc(O)c(O)c(O)c1 |
| | | CCCOC(═O)c1cc(O)c(O)c(O)c1 |
| | | Cc1cc(C(C)(C)C)c(O)c(C(C)(C)C)c1 |
| PG TBHQ | 1:3 | CCCOC(═O)c1cc(O)c(O)c(O)c1 |
| | | CC(C)(C)c1cc(O)ccc1O |
| | | CC(C)(C)c1cc(O)ccc1O |
| | | CC(C)(C)c1cc(O)ccc1O |
| PG TBHQ | 1:1 | CCCOC(═O)c1cc(O)c(O)c(O)c1 |
| | | CC(C)(C)c1cc(O)ccc1O |
| PG TBHQ | 3:1 | CCCOC(═O)c1cc(O)c(O)c(O)c1 |
| | | CCCOC(═O)c1cc(O)c(O)c(O)c1 |
| | | CCCOC(═O)c1cc(O)c(O)c(O)c1 |
| | | CC(C)(C)c1cc(O)ccc1O |
| PG Phenol | 1:3 | CCCOC(═O)c1cc(O)c(O)c(O)c1 |
| | | CC(C)(C)c1cc(CO)cc(C(C)(C)C)c1O |
| | | CC(C)(C)c1cc(CO)cc(C(C)(C)C)c1O |
| | | CC(C)(C)c1cc(CO)cc(C(C)(C)C)c1O |
| PG Phenol | 1:1 | CCCOC(═O)c1cc(O)c(O)c(O)c1 |
| | | CC(C)(C)c1cc(CO)cc(C(C)(C)C)c1O |
| PG Phenol | 3:1 | CCCOC(═O)c1cc(O)c(O)c(O)c1 |
| | | CCCOC(═O)c1cc(O)c(O)c(O)c1 |
| | | CCCOC(═O)c1cc(O)c(O)c(O)c1 |
| | | CC(C)(C)c1cc(CO)cc(C(C)(C)C)c1O |
| PG THBP | 1:3 | CCCOC(═O)c1cc(O)c(O)c(O)c1 |
| | | CCCC(═O)c1cc(O)c(O)cc1O |
| | | CCCC(═O)c1cc(O)c(O)cc1O |
| | | CCCC(═O)c1cc(O)c(O)cc1O |
| PG THBP | 1:1 | CCCOC(═O)c1cc(O)c(O)c(O)c1 |
| | | CCCC(═O)c1cc(O)c(O)cc1O |
| PG THBP | 3:1 | CCCOC(═O)c1cc(O)c(O)c(O)c1 |
| | | CCCOC(═O)c1cc(O)c(O)c(O)c1 |
| | | CCCOC(═O)c1cc(O)c(O)c(O)c1 |
| | | CCCC(═O)c1cc(O)c(O)cc1O |
| PG ETHO | 1:3 | CCCOC(═O)c1cc(O)c(O)c(O)c1 |
| | | CCOc1ccc2c(c1)C(C)═CC(C)(C)N2 |
| | | CCOc1ccc2c(c1)C(C)═CC(C)(C)N2 |
| | | CCOc1ccc2c(c1)C(C)═CC(C)(C)N2 |
| PG ETHO | 1:1 | CCCOC(═O)c1cc(O)c(O)c(O)c1 |
| | | CCOc1ccc2c(c1)C(C)═CC(C)(C)N2 |
| PG ETHO | 3:1 | CCCOC(═O)c1cc(O)c(O)c(O)c1 |
| | | CCCOC(═O)c1cc(O)c(O)c(O)c1 |
| | | CCCOC(═O)c1cc(O)c(O)c(O)c1 |
| | | CCOc1ccc2c(c1)C(C)═CC(C)(C)N2 |
| PG NDGA | 1:3 | CCCOC(═O)c1cc(O)c(O)c(O)c1 |
| | | CC(Cc1ccc(O)c(O)c1)C(C)Cc1ccc(O)c(O)c1 |
| | | CC(Cc1ccc(O)c(O)c1)C(C)Cc1ccc(O)c(O)c1 |
| | | CC(Cc1ccc(O)c(O)c1)C(C)Cc1ccc(O)c(O)c1 |
| PG NDGA | 1:1 | CCCOC(═O)c1cc(O)c(O)c(O)c1 |
| | | CC(Cc1ccc(O)c(O)c1)C(C)Cc1ccc(O)c(O)c1 |
| PG NDGA | 3:1 | CCCOC(═O)c1cc(O)c(O)c(O)c1 |
| | | CCCOC(═O)c1cc(O)c(O)c(O)c1 |
| | | CCCOC(═O)c1cc(O)c(O)c(O)c1 |
| | | CC(Cc1ccc(O)c(O)c1)C(C)Cc1ccc(O)c(O)c1 |
| PG LG | 3:1 | CCCOC(═O)c1cc(O)c(O)c(O)c1 |
| | | CCCOC(═O)c1cc(O)c(O)c(O)c1 |
| | | CCCOC(═O)c1cc(O)c(O)c(O)c1 |
| | | CCCCCCCCCCCCOC(═O)c1cc(O)c(O)c(O)c1 |
| PG OG | 1:3 | CCCOC(═O)c1cc(O)c(O)c(O)c1 |
| | | CCCCCCCCOC(═O)c1cc(O)c(O)c(O)c1 |
| | | CCCCCCCCOC(═O)c1cc(O)c(O)c(O)c1 |
| | | CCCCCCCCOC(═O)c1cc(O)c(O)c(O)c1 |
| PG OG | 1:1 | CCCOC(═O)c1cc(O)c(O)c(O)c1 |
| | | CCCCCCCCOC(═O)c1cc(O)c(O)c(O)c1 |
| PG OG | 3:1 | CCCOC(═O)c1cc(O)c(O)c(O)c1 |
| | | CCCOC(═O)c1cc(O)c(O)c(O)c1 |
| | | CCCOC(═O)c1cc(O)c(O)c(O)c1 |
| | | CCCCCCCCOC(═O)c1cc(O)c(O)c(O)c1 |
| BHA BHT | 1:3 | CCCC(CC)(CC)CC.COc1ccc(O)cc1 |
| | | Cc1cc(C(C)(C)C)c(O)c(C(C)(C)C)c1 |
| | | Cc1cc(C(C)(C)C)c(O)c(C(C)(C)C)c1 |
| | | Cc1cc(C(C)(C)C)c(O)c(C(C)(C)C)c1 |
| BHA BHT | 1:1 | CCCC(CC)(CC)CC.COc1ccc(O)cc1 |
| | | Cc1cc(C(C)(C)C)c(O)c(C(C)(C)C)c1 |
| BHA BHT | 3:1 | CCCC(CC)(CC)CC.COc1ccc(O)cc1 |
| | | CCCC(CC)(CC)CC.COc1ccc(O)cc1 |
| | | CCCC(CC)(CC)CC.COc1ccc(O)cc1 |
| | | Cc1cc(C(C)(C)C)c(O)c(C(C)(C)C)c1 |
| BHA TBHQ | 1:3 | CCCC(CC)(CC)CC.COc1ccc(O)cc1 |
| | | CC(C)(C)c1cc(O)ccc1O |
| | | CC(C)(C)c1cc(O)ccc1O |
| | | CC(C)(C)c1cc(O)ccc1O |
| BHA TBHQ | 1:1 | CCCC(CC)(CC)CC.COc1ccc(O)cc1 |
| | | CC(C)(C)c1cc(O)ccc1O |
| BHA TBHQ | 3:1 | CCCC(CC)(CC)CC.COc1ccc(O)cc1 |
| | | CCCC(CC)(CC)CC.COc1ccc(O)cc1 |
| | | CCCC(CC)(CC)CC.COc1ccc(O)cc1 |
| | | CC(C)(C)c1cc(O)ccc1O |
| BHA Phenol | 1:3 | CCCC(CC)(CC)CC.COc1ccc(O)cc1 |
| | | CC(C)(C)c1cc(CO)cc(C(C)(C)C)c1O |
| | | CC(C)(C)c1cc(CO)cc(C(C)(C)C)c1O |
| | | CC(C)(C)c1cc(CO)cc(C(C)(C)C)c1O |
| BHA Phenol | 1:1 | CCCC(CC)(CC)CC.COc1ccc(O)cc1 |
| | | CC(C)(C)c1cc(CO)cc(C(C)(C)C)c1O |
| BHA Phenol | 3:1 | CCCC(CC)(CC)CC.COc1ccc(O)cc1 |
| | | CCCC(CC)(CC)CC.COc1ccc(O)cc1 |
| | | CCCC(CC)(CC)CC.COc1ccc(O)cc1 |
| | | CC(C)(C)c1cc(CO)cc(C(C)(C)C)c1O |
| BHA THBP | 1:3 | CCCC(CC)(CC)CC.COc1ccc(O)cc1 |
| | | CCCC(═O)c1cc(O)c(O)cc1O |
| | | CCCC(═O)c1cc(O)c(O)cc1O |
| | | CCCC(═O)c1cc(O)c(O)cc1O |
| BHA THBP | 1:1 | CCCC(CC)(CC)CC.COc1ccc(O)cc1 |
| | | CCCC(═O)c1cc(O)c(O)cc1O |
| BHA THBP | 3:1 | CCCC(CC)(CC)CC.COc1ccc(O)cc1 |
| | | CCCC(CC)(CC)CC.COc1ccc(O)cc1 |
| | | CCCC(CC)(CC)CC.COc1ccc(O)cc1 |
| | | CCCC(═O)c1cc(O)c(O)cc1O |
| BHA ETHO | 1:3 | CCCC(CC)(CC)CC.COc1ccc(O)cc1 |
| | | CCOc1ccc2c(c1)C(C)═CC(C)(C)N2 |
| | | CCOc1ccc2c(c1)C(C)═CC(C)(C)N2 |
| | | CCOc1ccc2c(c1)C(C)═CC(C)(C)N2 |
| BHA ETHO | 1:1 | CCCC(CC)(CC)CC.COc1ccc(O)cc1 |
| | | CCOc1ccc2c(c1)C(C)═CC(C)(C)N2 |
| BHA ETHO | 3:1 | CCCC(CC)(CC)CC.COc1ccc(O)cc1 |
| | | CCCC(CC)(CC)CC.COc1ccc(O)cc1 |
| | | CCCC(CC)(CC)CC.COc1ccc(O)cc1 |
| | | CCOc1ccc2c(c1)C(C)═CC(C)(C)N2 |
| BHA NDGA | 1:3 | CCCC(CC)(CC)CC.COc1ccc(O)cc1 |
| | | CC(Cc1ccc(O)c(O)c1)C(C)Cc1ccc(O)c(O)c1 |
| | | CC(Cc1ccc(O)c(O)c1)C(C)Cc1ccc(O)c(O)c1 |
| | | CC(Cc1ccc(O)c(O)c1)C(C)Cc1ccc(O)c(O)c1 |
| BHA NDGA | 1:1 | CCCC(CC)(CC)CC.COc1ccc(O)cc1 |
| | | CC(Cc1ccc(O)c(O)c1)C(C)Cc1ccc(O)c(O)c1 |
| BHA NDGA | 3:1 | CCCC(CC)(CC)CC.COc1ccc(O)cc1 |
| | | CCCC(CC)(CC)CC.COc1ccc(O)cc1 |
| | | CCCC(CC)(CC)CC.COc1ccc(O)cc1 |
| | | CC(Cc1ccc(O)c(O)c1)C(C)Cc1ccc(O)c(O)c1 |
| BHA LG | 1:3 | CCCC(CC)(CC)CC.COc1ccc(O)cc1 |
| | | CCCCCCCCCCCCOC(═O)c1cc(O)c(O)c(O)c1 |
| | | CCCCCCCCCCCCOC(═O)c1cc(O)c(O)c(O)c1 |
| | | CCCCCCCCCCCCOC(═O)c1cc(O)c(O)c(O)c1 |
| BHA LG | 1:1 | CCCC(CC)(CC)CC.COc1ccc(O)cc1 |
| | | CCCCCCCCCCCCOC(═O)c1cc(O)c(O)c(O)c1 |
| BHA LG | 3:1 | CCCC(CC)(CC)CC.COc1ccc(O)cc1 |
| | | CCCC(CC)(CC)CC.COc1ccc(O)cc1 |
| | | CCCC(CC)(CC)CC.COc1ccc(O)cc1 |
| | | CCCCCCCCCCCCOC(═O)c1cc(O)c(O)c(O)c1 |
| BHA OG | 1:3 | CCCC(CC)(CC)CC.COc1ccc(O)cc1 |
| | | CCCCCCCCOC(═O)c1cc(O)c(O)c(O)c1 |
| | | CCCCCCCCOC(═O)c1cc(O)c(O)c(O)c1 |
| | | CCCCCCCCOC(═O)c1cc(O)c(O)c(O)c1 |
| BHA OG | 1:1 | CCCC(CC)(CC)CC.COc1ccc(O)cc1 |
| | | CCCCCCCCOC(═O)c1cc(O)c(O)c(O)c1 |
| BHA OG | 3:1 | CCCC(CC)(CC)CC.COc1ccc(O)cc1 |
| | | CCCC(CC)(CC)CC.COc1ccc(O)cc1 |
| | | CCCC(CC)(CC)CC.COc1ccc(O)cc1 |
| | | CCCCCCCCOC(═O)c1cc(O)c(O)c(O)c1 |
| BHT TBHQ | 1:3 | Cc1cc(C(C)(C)C)c(O)c(C(C)(C)C)c1 |
| | | CC(C)(C)c1cc(O)ccc1O |
| | | CC(C)(C)c1cc(O)ccc1O |
| | | CC(C)(C)c1cc(O)ccc1O |
| BHT TBHQ | 1:1 | Cc1cc(C(C)(C)C)c(O)c(C(C)(C)C)c1 |
| | | CC(C)(C)c1cc(O)ccc1O |
| BHT TBHQ | 3:1 | Cc1cc(C(C)(C)C)c(O)c(C(C)(C)C)c1 |
| | | Cc1cc(C(C)(C)C)c(O)c(C(C)(C)C)c1 |
| | | Cc1cc(C(C)(C)C)c(O)c(C(C)(C)C)c1 |
| | | CC(C)(C)c1cc(O)ccc1O |
| BHT Phenol | 1:3 | Cc1cc(C(C)(C)C)c(O)c(C(C)(C)C)c1 |
| | | CC(C)(C)c1cc(CO)cc(C(C)(C)C)c1O |
| | | CC(C)(C)c1cc(CO)cc(C(C)(C)C)c1O |
| | | CC(C)(C)c1cc(CO)cc(C(C)(C)C)c1O |
| BHT Phenol | 1:1 | Cc1cc(C(C)(C)C)c(O)c(C(C)(C)C)c1 |
| | | CC(C)(C)c1cc(CO)cc(C(C)(C)C)c1O |
| BHT Phenol | 3:1 | Cc1cc(C(C)(C)C)c(O)c(C(C)(C)C)c1 |
| | | Cc1cc(C(C)(C)C)c(O)c(C(C)(C)C)c1 |
| | | Cc1cc(C(C)(C)C)c(O)c(C(C)(C)C)c1 |
| | | CC(C)(C)c1cc(CO)cc(C(C)(C)C)c1O |
| BHT THBP | 1:3 | Cc1cc(C(C)(C)C)c(O)c(C(C)(C)C)c1 |
| | | CCCC(═O)c1cc(O)c(O)cc1O |
| | | CCCC(═O)c1cc(O)c(O)cc1O |
| | | CCCC(═O)c1cc(O)c(O)cc1O |
| BHT THBP | 1:1 | Cc1cc(C(C)(C)C)c(O)c(C(C)(C)C)c1 |
| | | CCCC(═O)c1cc(O)c(O)cc1O |
| BHT THBP | 3:1 | Cc1cc(C(C)(C)C)c(O)c(C(C)(C)C)c1 |
| | | Cc1cc(C(C)(C)C)c(O)c(C(C)(C)C)c1 |
| | | Cc1cc(C(C)(C)C)c(O)c(C(C)(C)C)c1 |
| | | CCCC(═O)c1cc(O)c(O)cc1O |
| BHT ETHO | 1:3 | Cc1cc(C(C)(C)C)c(O)c(C(C)(C)C)c1 |
| | | CCOc1ccc2c(c1)C(C)═CC(C)(C)N2 |
| | | CCOc1ccc2c(c1)C(C)═CC(C)(C)N2 |
| | | CCOc1ccc2c(c1)C(C)═CC(C)(C)N2 |
| BHT ETHO | 1:1 | Cc1cc(C(C)(C)C)c(O)c(C(C)(C)C)c1 |
| | | CCOc1ccc2c(c1)C(C)═CC(C)(C)N2 |
| BHT ETHO | 3:1 | Cc1cc(C(C)(C)C)c(O)c(C(C)(C)C)c1 |
| | | Cc1cc(C(C)(C)C)c(O)c(C(C)(C)C)c1 |
| | | Cc1cc(C(C)(C)C)c(O)c(C(C)(C)C)c1 |
| | | CCOc1ccc2c(c1)C(C)═CC(C)(C)N2 |
| BHT NDGA | 1:3 | Cc1cc(C(C)(C)C)c(O)c(C(C)(C)C)c1 |
| | | CC(Cc1ccc(O)c(O)c1)C(C)Cc1ccc(O)c(O)c1 |
| | | CC(Cc1ccc(O)c(O)c1)C(C)Cc1ccc(O)c(O)c1 |
| | | CC(Cc1ccc(O)c(O)c1)C(C)Cc1ccc(O)c(O)c1 |
| BHT NDGA | 1:1 | Cc1cc(C(C)(C)C)c(O)c(C(C)(C)C)c1 |
| | | CC(Cc1ccc(O)c(O)c1)C(C)Cc1ccc(O)c(O)c1 |
| BHT NDGA | 3:1 | Cc1cc(C(C)(C)C)c(O)c(C(C)(C)C)c1 |
| | | Cc1cc(C(C)(C)C)c(O)c(C(C)(C)C)c1 |
| | | Cc1cc(C(C)(C)C)c(O)c(C(C)(C)C)c1 |
| | | CC(Cc1ccc(O)c(O)c1)C(C)Cc1ccc(O)c(O)c1 |
| | | BHT LG 1:3 |
| | | Cc1cc(C(C)(C)C)c(O)c(C(C)(C)C)c1 |
| | | CCCCCCCCCCCCOC(═O)c1cc(O)c(O)c(O)c1 |
| BHT LG | 1:1 | Cc1cc(C(C)(C)C)c(O)c(C(C)(C)C)c1 |
| | | CCCCCCCCCCCCOC(═O)c1cc(O)c(O)c(O)c1 |
| BHT LG | 3:1 | Cc1cc(C(C)(C)C)c(O)c(C(C)(C)C)c1 |
| | | Cc1cc(C(C)(C)C)c(O)c(C(C)(C)C)c1 |
| | | Cc1cc(C(C)(C)C)c(O)c(C(C)(C)C)c1 |
| | | CCCCCCCCCCCCOC(═O)c1cc(O)c(O)c(O)c1 |
| BHT OG | 1:3 | Cc1cc(C(C)(C)C)c(O)c(C(C)(C)C)c1 |
| | | CCCCCCCCOC(═O)c1cc(O)c(O)c(O)c1 |
| | | CCCCCCCCOC(═O)c1cc(O)c(O)c(O)c1 |
| | | CCCCCCCCOC(═O)c1cc(O)c(O)c(O)c1 |
| BHT OG | 1:1 | Cc1cc(C(C)(C)C)c(O)c(C(C)(C)C)c1 |
| | | CCCCCCCCOC(═O)c1cc(O)c(O)c(O)c1 |
| BHT OG | 3:1 | Cc1cc(C(C)(C)C)c(O)c(C(C)(C)C)c1 |
| | | Cc1cc(C(C)(C)C)c(O)c(C(C)(C)C)c1 |
| | | Cc1cc(C(C)(C)C)c(O)c(C(C)(C)C)c1 |
| | | CCCCCCCCOC(═O)c1cc(O)c(O)c(O)c1 |
| TBHQ | 1:3 | CC(C)(C)c1cc(O)ccc1O |
| Phenol | | CC(C)(C)c1cc(CO)cc(C(C)(C)C)c1O |
| | | CC(C)(C)c1cc(CO)cc(C(C)(C)C)c1O |
| | | CC(C)(C)c1cc(CO)cc(C(C)(C)C)c1O |
| TBHQ | 1:1 | CC(C)(C)c1cc(O)ccc1O |
| Phenol | | CC(C)(C)c1cc(CO)cc(C(C)(C)C)c1O |
| TBHQ | 3:1 | CC(C)(C)c1cc(O)ccc1O |
| Phenol | | CC(C)(C)c1cc(O)ccc1O |
| | | CC(C)(C)c1cc(O)ccc1O |
| | | CC(C)(C)c1cc(CO)cc(C(C)(C)C)c1O |
| TBHQ | 1:3 | CC(C)(C)c1cc(O)ccc1O |
| THBP | | CCCC(═O)c1cc(O)c(O)cc1O |
| | | CCCC(═O)c1cc(O)c(O)cc1O |
| | | CCCC(═O)c1cc(O)c(O)cc1O |
| TBHQ | 1:1 | CC(C)(C)c1cc(O)ccc1O |
| THBP | | CCCC(═O)c1cc(O)c(O)cc1O |
| TBHQ | 3:1 | CC(C)(C)c1cc(O)ccc1O |
| THBP | | CC(C)(C)c1cc(O)ccc1O |
| | | CC(C)(C)c1cc(O)ccc1O |
| | | CCCC(═O)c1cc(O)c(O)cc1O |
| TBHQ | 1:3 | CC(C)(C)c1cc(O)ccc1O |
| ETHO | | CCOc1ccc2c(c1)C(C)═CC(C)(C)N2 |
| | | CCOc1ccc2c(c1)C(C)═CC(C)(C)N2 |
| | | CCOc1ccc2c(c1)C(C)═CC(C)(C)N2 |
| TBHQ | 1:1 | CC(C)(C)c1cc(O)ccc1O |
| ETHO | | CCOc1ccc2c(c1)C(C)═CC(C)(C)N2 |
| TBHQ | 3:1 | CC(C)(C)c1cc(O)ccc1O |
| ETHO | | CC(C)(C)c1cc(O)ccc1O |
| | | CC(C)(C)c1cc(O)ccc1O |
| | | CCOc1ccc2c(c1)C(C)═CC(C)(C)N2 |
| TBHQ | 1:3 | CC(C)(C)c1cc(O)ccc1O |
| NDGA | | CC(Cc1ccc(O)c(O)c1)C(C)Cc1ccc(O)c(O)c1 |
| | | CC(Cc1ccc(O)c(O)c1)C(C)Cc1ccc(O)c(O)c1 |
| | | CC(Cc1ccc(O)c(O)c1)C(C)Cc1ccc(O)c(O)c1 |
| TBHQ | 1:1 | CC(C)(C)c1cc(O)ccc1O |
| NDGA | | CC(Cc1ccc(O)c(O)c1)C(C)Cc1ccc(O)c(O)c1 |
| TBHQ | 3:1 | CC(C)(C)c1cc(O)ccc1O |
| NDGA | | CC(C)(C)c1cc(O)ccc1O |
| | | CC(C)(C)c1cc(O)ccc1O |
| | | CC(Cc1ccc(O)c(O)c1)C(C)Cc1ccc(O)c(O)c1 |
| TBHQ LG | 1:3 | CC(C)(C)c1cc(O)ccc1O |
| | | CCCCCCCCCCCCOC(═O)c1cc(O)c(O)c(O)c1 |
| | | CCCCCCCCCCCCOC(═O)c1cc(O)c(O)c(O)c1 |
| | | CCCCCCCCCCCCOC(═O)c1cc(O)c(O)c(O)c1 |
| TBHQ LG | 1:1 | CC(C)(C)c1cc(O)ccc1O |
| | | CCCCCCCCCCCCOC(═O)c1cc(O)c(O)c(O)c1 |
| TBHQ LG | 3:1 | CC(C)(C)c1cc(O)ccc1O |
| | | CC(C)(C)c1cc(O)ccc1O |
| | | CC(C)(C)c1cc(O)ccc1O |
| | | CCCCCCCCCCCCOC(═O)c1cc(O)c(O)c(O)c1 |
| TBHQ OG | 1:3 | CC(C)(C)c1cc(O)ccc1O |
| | | CCCCCCCCOC(═O)c1cc(O)c(O)c(O)c1 |
| | | CCCCCCCCOC(═O)c1cc(O)c(O)c(O)c1 |
| | | CCCCCCCCOC(═O)c1cc(O)c(O)c(O)c1 |
| TBHQ OG | 1:1 | CC(C)(C)c1cc(O)ccc1O |
| | | CCCCCCCCOC(═O)c1cc(O)c(O)c(O)c1 |
| TBHQ OG | 3:1 | CC(C)(C)c1cc(O)ccc1O |
| | | CC(C)(C)c1cc(O)ccc1O |
| | | CC(C)(C)c1cc(O)ccc1O |
| | | CCCCCCCCOC(═O)c1cc(O)c(O)c(O)c1 |
| Phenol | 1:3 | CC(C)(C)c1cc(CO)cc(C(C)(C)C)c1O |
| THBP | | CCCC(═O)c1cc(O)c(O)cc1O |
| | | CCCC(═O)c1cc(O)c(O)cc1O |
| | | CCCC(═O)c1cc(O)c(O)cc1O |
| Phenol | 1:1 | CC(C)(C)c1cc(CO)cc(C(C)(C)C)c1O |
| THBP | | CCCC(═O)c1cc(O)c(O)cc1O |
| Phenol | 3:1 | CC(C)(C)c1cc(CO)cc(C(C)(C)C)c1O |
| THBP | | CC(C)(C)c1cc(CO)cc(C(C)(C)C)c1O |
| | | CC(C)(C)c1cc(CO)cc(C(C)(C)C)c1O |
| | | CCCC(═O)c1cc(O)c(O)cc1O |
| Phenol | 1:3 | CC(C)(C)c1cc(CO)cc(C(C)(C)C)c1O |
| ETHO | | CCOc1ccc2c(c1)C(C)═CC(C)(C)N2 |
| | | CCOc1ccc2c(c1)C(C)═CC(C)(C)N2 |
| | | CCOc1ccc2c(c1)C(C)═CC(C)(C)N2 |
| Phenol | 1:1 | CC(C)(C)c1cc(CO)cc(C(C)(C)C)c1O |
| ETHO | | CCOc1ccc2c(c1)C(C)═CC(C)(C)N2 |
| Phenol | 3:1 | CC(C)(C)c1cc(CO)cc(C(C)(C)C)c1O |
| ETHO | | CC(C)(C)c1cc(CO)cc(C(C)(C)C)c1O |
| | | CC(C)(C)c1cc(CO)cc(C(C)(C)C)c1O |
| | | CCOc1ccc2c(c1)C(C)═CC(C)(C)N2 |
| Phenol | 1:3 | CC(C)(C)c1cc(CO)cc(C(C)(C)C)c1O |
| NDGA | | CC(Cc1ccc(O)c(O)c1)C(C)Cc1ccc(O)c(O)c1 |
| | | CC(Cc1ccc(O)c(O)c1)C(C)Cc1ccc(O)c(O)c1 |
| | | CC(Cc1ccc(O)c(O)c1)C(C)Cc1ccc(O)c(O)c1 |
| Phenol | 1:1 | CC(C)(C)c1cc(CO)cc(C(C)(C)C)c1O |
| NDGA | | CC(Cc1ccc(O)c(O)c1)C(C)Cc1ccc(O)c(O)c1 |
| Phenol | 3:1 | CC(C)(C)c1cc(CO)cc(C(C)(C)C)c1O |
| NDGA | | CC(C)(C)c1cc(CO)cc(C(C)(C)C)c1O |
| | | CC(C)(C)c1cc(CO)cc(C(C)(C)C)c1O |
| | | CC(Cc1ccc(O)c(O)c1)C(C)Cc1ccc(O)c(O)c1 |
| Phenol LG | 1:3 | CC(C)(C)c1cc(CO)cc(C(C)(C)C)c1O |
| | | CCCCCCCCCCCCOC(═O)c1cc(O)c(O)c(O)c1 |
| | | CCCCCCCCCCCCOC(═O)c1cc(O)c(O)c(O)c1 |
| | | CCCCCCCCCCCCOC(═O)c1cc(O)c(O)c(O)c1 |
| Phenol LG | 1:1 | CC(C)(C)c1cc(CO)cc(C(C)(C)C)c1O |
| | | CCCCCCCCCCCCOC(═O)c1cc(O)c(O)c(O)c1 |
| Phenol LG | 3:1 | CC(C)(C)c1cc(CO)cc(C(C)(C)C)c1O |
| | | CC(C)(C)c1cc(CO)cc(C(C)(C)C)c1O |
| | | CC(C)(C)c1cc(CO)cc(C(C)(C)C)c1O |
| | | CCCCCCCCCCCCOC(═O)c1cc(O)c(O)c(O)c1 |
| Phenol OG | 1:3 | CC(C)(C)c1cc(CO)cc(C(C)(C)C)c1O |
| | | CCCCCCCCOC(═O)c1cc(O)c(O)c(O)c1 |
| | | CCCCCCCCOC(═O)c1cc(O)c(O)c(O)c1 |
| | | CCCCCCCCOC(═O)c1cc(O)c(O)c(O)c1 |
| Phenol OG | 1:1 | CC(C)(C)c1cc(CO)cc(C(C)(C)C)c1O |
| | | CCCCCCCCOC(═O)c1cc(O)c(O)c(O)c1 |
| Phenol OG | 3:1 | CC(C)(C)c1cc(CO)cc(C(C)(C)C)c1O |
| | | CC(C)(C)c1cc(CO)cc(C(C)(C)C)c1O |
| | | CC(C)(C)c1cc(CO)cc(C(C)(C)C)c1O |
| | | CCCCCCCCOC(═O)c1cc(O)c(O)c(O)c1 |
| THBP | 1:3 | CCCC(═O)c1cc(O)c(O)cc1O |
| ETHO | | CCOc1ccc2c(c1)C(C)═CC(C)(C)N2 |
| | | CCOc1ccc2c(c1)C(C)═CC(C)(C)N2 |
| | | CCOc1ccc2c(c1)C(C)═CC(C)(C)N2 |
| THBP | 1:1 | CCCC(═O)c1cc(O)c(O)cc1O |
| ETHO | | CCOc1ccc2c(c1)C(C)═CC(C)(C)N2 |
| THBP | 3:1 | CCCC(═O)c1cc(O)c(O)cc1O |
| THO | | CCCC(═O)c1cc(O)c(O)cc1O |
| | | CCCC(═O)c1cc(O)c(O)cc1O |
| | | CCOc1ccc2c(c1)C(C)═CC(C)(C)N2 |
| THBP | 1:3 | CCCC(═O)c1cc(O)c(O)cc1O |
| NDGA | | CC(Cc1ccc(O)c(O)c1)C(C)Cc1ccc(O)c(O)c1 |
| | | CC(Cc1ccc(O)c(O)c1)C(C)Cc1ccc(O)c(O)c1 |
| | | CC(Cc1ccc(O)c(O)c1)C(C)Cc1ccc(O)c(O)c1 |
| THBP | 1:1 | CCCC(═O)c1cc(O)c(O)cc1O |
| NDGA | | CC(Cc1ccc(O)c(O)c1)C(C)Cc1ccc(O)c(O)c1 |
| THBP | 3:1 | CCCC(═O)c1cc(O)c(O)cc1O |
| NDGA | | CCCC(═O)c1cc(O)c(O)cc1O |
| | | CCCC(═O)c1cc(O)c(O)cc1O |
| | | CC(Cc1ccc(O)c(O)c1)C(C)Cc1ccc(O)c(O)c1 |
| THBP LG | 1:3 | CCCC(═O)c1cc(O)c(O)cc1O |
| | | CCCCCCCCCCCCOC(═O)c1cc(O)c(O)c(O)c1 |
| | | CCCCCCCCCCCCOC(═O)c1cc(O)c(O)c(O)c1 |
| | | CCCCCCCCCCCCOC(═O)c1cc(O)c(O)c(O)c1 |
| THBP LG | 1:1 | CCCC(═O)c1cc(O)c(O)cc1O |
| | | CCCCCCCCCCCCOC(═O)c1cc(O)c(O)c(O)c1 |
| THBP LG | 3:1 | CCCC(═O)c1cc(O)c(O)cc1O |
| | | CCCC(═O)c1cc(O)c(O)cc1O |
| | | CCCC(═O)c1cc(O)c(O)cc1O |
| | | CCCCCCCCCCCCOC(═O)c1cc(O)c(O)c(O)c1 |
| THBP OG | 1:3 | CCCC(═O)c1cc(O)c(O)cc1O |
| | | CCCCCCCCOC(═O)c1cc(O)c(O)c(O)c1 |
| | | CCCCCCCCOC(═O)c1cc(O)c(O)c(O)c1 |
| | | CCCCCCCCOC(═O)c1cc(O)c(O)c(O)c1 |
| THBP OG | 1:1 | CCCC(═O)c1cc(O)c(O)cc1O |
| | | CCCCCCCCOC(═O)c1cc(O)c(O)c(O)c1 |
| THBP OG | 3:1 | CCCC(═O)c1cc(O)c(O)cc1O |
| | | CCCC(═O)c1cc(O)c(O)cc1O |
| | | CCCC(═O)c1cc(O)c(O)cc1O |
| | | CCCCCCCCOC(═O)c1cc(O)c(O)c(O)c1 |
| ETHO | 1:3 | CCOc1ccc2c(c1)C(C)═CC(C)(C)N2 |
| NDGA | | CC(Cc1ccc(O)c(O)c1)C(C)Cc1ccc(O)c(O)c1 |
| | | CC(Cc1ccc(O)c(O)c1)C(C)Cc1ccc(O)c(O)c1 |
| | | CC(Cc1ccc(O)c(O)c1)C(C)Cc1ccc(O)c(O)c1 |
| ETHO | 1:1 | CCOc1ccc2c(c1)C(C)═CC(C)(C)N2 |
| NDGA | | CC(Cc1ccc(O)c(O)c1)C(C)Cc1ccc(O)c(O)c1 |
| ETHO | 3:1 | CCOc1ccc2c(c1)C(C)═CC(C)(C)N2 |
| NDGA | | CCOc1ccc2c(c1)C(C)═CC(C)(C)N2 |
| | | CCOc1ccc2c(c1)C(C)═CC(C)(C)N2 |
| | | CC(Cc1ccc(O)c(O)c1)C(C)Cc1ccc(O)c(O)c1 |
| ETHO LG | 1:3 | CCOc1ccc2c(c1)C(C)═CC(C)(C)N2 |
| | | CCCCCCCCCCCCOC(═O)c1cc(O)c(O)c(O)c1 |
| | | CCCCCCCCCCCCOC(═O)c1cc(O)c(O)c(O)c1 |
| | | CCCCCCCCCCCCOC(═O)c1cc(O)c(O)c(O)c1 |
| ETHO LG | 1:1 | CCOc1ccc2c(c1)C(C)═CC(C)(C)N2 |
| | | CCCCCCCCCCCCOC(═O)c1cc(O)c(O)c(O)c1 |
| ETHO LG | 3:1 | CCOc1ccc2c(c1)C(C)═CC(C)(C)N2 |
| | | CCOc1ccc2c(c1)C(C)═CC(C)(C)N2 |
| | | CCOc1ccc2c(c1)C(C)═CC(C)(C)N2 |
| | | CCCCCCCCCCCCOC(═O)c1cc(O)c(O)c(O)c1 |
| ETHO OG | 1:3 | CCOc1ccc2c(c1)C(C)═CC(C)(C)N2 |
| | | CCCCCCCCOC(═O)c1cc(O)c(O)c(O)c1 |
| | | CCCCCCCCOC(═O)c1cc(O)c(O)c(O)c1 |
| | | CCCCCCCCOC(═O)c1cc(O)c(O)c(O)c1 |
| ETHO OG | 1:1 | CCOc1ccc2c(c1)C(C)═CC(C)(C)N2 |
| | | CCCCCCCCOC(═O)c1cc(O)c(O)c(O)c1 |
| ETHO OG | 3:1 | CCOc1ccc2c(c1)C(C)═CC(C)(C)N2 |
| | | CCOc1ccc2c(c1)C(C)═CC(C)(C)N2 |
| | | CCOc1ccc2c(c1)C(C)═CC(C)(C)N2 |
| | | CCCCCCCCOC(═O)c1cc(O)c(O)c(O)c1 |
| NDGA LG | 1:3 | CC(Cc1ccc(O)c(O)c1)C(C)Cc1ccc(O)c(O)c1 |
| | | CCCCCCCCCCCCOC(═O)c1cc(O)c(O)c(O)c1 |
| | | CCCCCCCCCCCCOC(═O)c1cc(O)c(O)c(O)c1 |
| | | CCCCCCCCCCCCOC(═O)c1cc(O)c(O)c(O)c1 |
| NDGA LG | 1:1 | CC(Cc1ccc(O)c(O)c1)C(C)Cc1ccc(O)c(O)c1 |
| | | CCCCCCCCCCCCOC(═O)c1cc(O)c(O)c(O)c1 |
| NDGA LG | 3:1 | CC(Cc1ccc(O)c(O)c1)C(C)Cc1ccc(O)c(O)c1 |
| | | CC(Cc1ccc(O)c(O)c1)C(C)Cc1ccc(O)c(O)c1 |
| | | CC(Cc1ccc(O)c(O)c1)C(C)Cc1ccc(O)c(O)c1 |
| | | CCCCCCCCCCCCOC(═O)c1cc(O)c(O)c(O)c1 |
| NDGA OG | 1:3 | CC(Cc1ccc(O)c(O)c1)C(C)Cc1ccc(O)c(O)c1 |
| | | CCCCCCCCOC(═O)c1cc(O)c(O)c(O)c1 |
| | | CCCCCCCCOC(═O)c1cc(O)c(O)c(O)c1 |
| | | CCCCCCCCOC(═O)c1cc(O)c(O)c(O)c1 |
| NDGA OG | 1:1 | CC(Cc1ccc(O)c(O)c1)C(C)Cc1ccc(O)c(O)c1 |
| | | CCCCCCCCOC(═O)c1cc(O)c(O)c(O)c1 |
| NDGA OG | 3:1 | CC(Cc1ccc(O)c(O)c1)C(C)Cc1ccc(O)c(O)c1 |
| | | CC(Cc1ccc(O)c(O)c1)C(C)Cc1ccc(O)c(O)c1 |
| | | CC(Cc1ccc(O)c(O)c1)C(C)Cc1ccc(O)c(O)c1 |
| | | CCCCCCCCOC(═O)c1cc(O)c(O)c(O)c1 |
| LG OG | 1:3 | CCCCCCCCCCCCOC(═O)c1cc(O)c(O)c(O)c1 |
| | | CCCCCCCCOC(═O)c1cc(O)c(O)c(O)c1 |
| | | CCCCCCCCOC(═O)c1cc(O)c(O)c(O)c1 |
| | | CCCCCCCCOC(═O)c1cc(O)c(O)c(O)c1 |
| LG OG | 1:1 | CCCCCCCCCCCCOC(═O)c1cc(O)c(O)c(O)c1 |
| | | CCCCCCCCOC(═O)c1cc(O)c(O)c(O)c1 |
| LG OG | 3:1 | CCCCCCCCCCCCOC(═O)c1cc(O)c(O)c(O)c1 |
| | | CCCCCCCCCCCCOC(═O)c1cc(O)c(O)c(O)c1 |
| | | CCCCCCCCCCCCOC(═O)c1cc(O)c(O)c(O)c1 |
| | | CCCCCCCCOC(═O)c1cc(O)c(O)c(O)c1 |

In one embodiment, the system uses a language-based algorithm, pre-trained using general unlabeled chemical data. This algorithm is fined-tuned as a regressor using an existing database of antioxidant mixtures. This transformer-based neural network model is trained to recognize the patterns in SMILES strings of a sequence of mixtures in a database and predict the corresponding CI values (or other available metric), from which the antioxidant mixtures can be classified as synergistic, additive, or antagonistic.

In one embodiment, the system uses molecular fingerprints (vectorized representations of molecules capturing precise details of atomic configurations, as shown in FIG. 9. Unlike text-based representations of chemical structures, molecular fingerprints are derived from molecular graphs, enabling calculations based on global molecular descriptors, feature position-aware encoding of individual atom and bond features, and preserve the chemical identity of functional groups. Thus, the database can be augmented using a cheminformatics toolkit, such as RDKit which is an open-source cheminformatics software toolkit designed for working with small molecules. Toward this goal, all the hydrogen bond donor and acceptor molecules will be first converted (from their chemical name) into canonical SMILES using PubChemPy (v.1.0.4, pubchempy.readthedocs.io/).

Then, the resulting representation of each component on each mixture reported were used to expand the database through the open-source RDKit Python (v.2022.03.3) application programming interface (API). Potentially relevant chemical descriptors pertinent to the context of hydrogen bonding would be selected including a number of atoms, number of heavy atoms, polar surface area, molecular weight, number of aromatic rings, number of heteroatoms, logP, number of carbon atoms, number of oxygen atoms, number of nitrogen atoms, and number of chloride atoms. In this embodiment, the hydrogen bond donor count and hydrogen bond acceptor count can be queried using PubChemPy, due to reported inconsistencies between the values retrieved by RDKit with respect to those published in the literature. The improvement over the existing technology can include vectorization being accomplished prior to the training process, allowing the system to simultaneously consider all the descriptors and features, thus speeding up the overall learning. The molecules presented to the algorithm retain their molecular structure, allowing calculations in both forward and backward directions. Therefore, the geometry and characteristics of other antioxidants (or adjuvants) can be predicted and then searched for using similarity approaches. This system allows finding non-linear relationships in relatively large datasets. The use of vectors enables building feature maps, rendering a score (in the last layer) that indicates the degree of molecular overlap between the selected structures. The score can also be used to discover new synergistic combinations based on geometry.

The system can integrate a multi-dimensional deep vector model and critically assess its performance against other models (e.g., transformer-based models shown in FIGS. 1A and 1B). The system can integrate multiple variables as inputs for the model. In order to train the system a portion of the data (around 80% of the total database, randomly selected) was used as a training dataset. The remaining 15% of the database will be used to test the system's accuracy, while the remaining 5% will be reserved to fine-tune the hyperparameter of the model (evaluation dataset). This process will be assessed following the loss parameter and considering the computational resources required.

The system can then produce a database of mixtures of antioxidants, ranked according to their probability of displaying synergistic effects. The system predictions (generated from fingerprints, vectors including selected features) to be far more accurate than those obtained with the existing language-based model. As a sample of the improved capabilities of the proposed vector model displays the CI value (predicted with a non-optimized model) as a function of the experimental ΔTBARS.

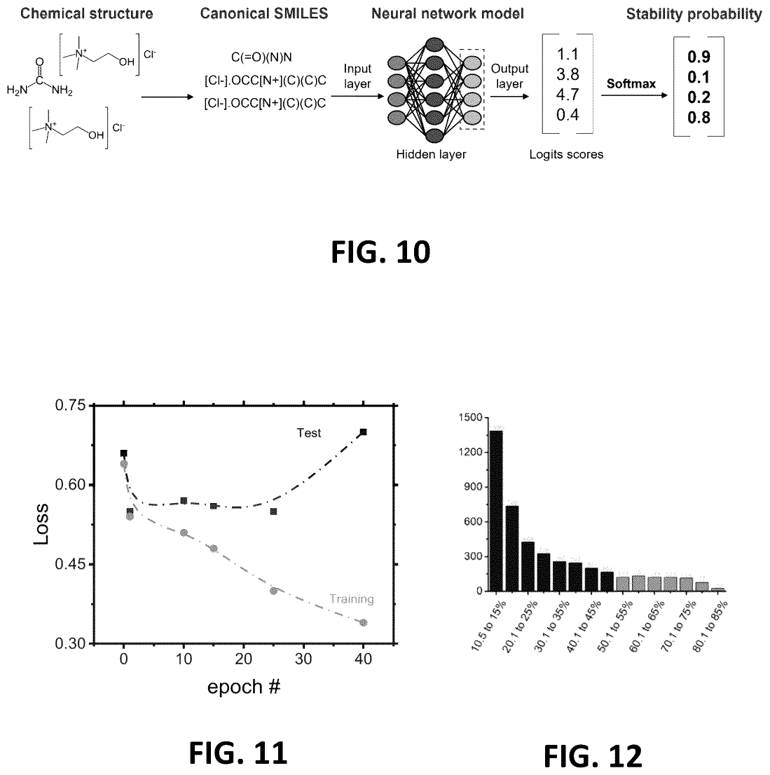

In one embodiment, the system will first apply an existing complementary algorithm, pre-trained using general unlabeled chemical data and fined tuned as a binary classifier using an existing DES/NADES database. This transformer-based neural network model is trained to recognize the patterns in strings that lead to the formation of either stable DES/NADES or simple mixtures of compounds not leading to the formation of stable DES/NADES (binary classification). This can be accomplished by implementing a SoftMax function on the raw output of the last layer from the deep neural network model. Scores will be then postprocessed and in a compound finder module, that allows filtering and ranking the output according to their probability to form a stable DES/NADES. A summary of the proposed strategy is shown in FIG. 1A and FIG. 10.

From there, the system can understand hydrogen bonding in the context of the formation of DES.

In one embodiment, the system can include modification of the databases such as adding entries to our existing DES/NADES database (e.g., 1200 entries) following the procedure described herein; but searching third party sources describing the formation of DES/NADES. The focus will be on non-toxic components that can be used to generate NADES for human consumption. This consideration also means that we will exclude components from our database (methanol, ethanolamine, tetrabutylammonium bromide, etc.) that are not generally regarded as safe (for instance, according to the US-FDA). Toward that goal, we have already retrieved an extensive DES database published by Sadeghi and a database of chiral DES published by Hopkins. The stoichiometric ratios of the reported DES/NADES were retrieved from those manuscripts using a python script, leading to a data frame containing the name of the hydrogen bond acceptor, the name of the hydrogen bond donor, the molar ratio, and (if reported) the corresponding melting point, density, viscosity, conductivity, surface tension, and refractive index. These databases were stored in a csv file and have added approximately 2500 new examples of binary and tertiary DES/NADES.

In one embodiment, the database will be augmented using RDKit, following the procedure described herein. This process will generate the required vectorized representations of the antioxidants, required for the advanced model.

In one embodiment, the system will include development of a multi-dimensional deep vector model and critically assess its performance against experimental and literature-based information. One of the possible tests to be performed to optimize the performance of the algorithm, FIG. 11 shows the dependence of the loss function as a function of the epoch #, for the training and test datasets. In this example, the classifier trained with <5 epochs was underfitted, indicating that the model was not yet able to get meaningful information from the chemical space. On the other hand, the maximum difference was obtained at epoch #40, where the loss function for the test dataset suddenly increases, suggesting that the system can be overfitted. In this context, an optimum classifier should be trained with the number of epochs in between these two extremes, where the number of interactions is enough to learn important information from the dataset but not enough to overfit by “memorizing” noise and other useful information in the training data. Also following the general approach described herein this system can implement a number of strategies to identify and address accuracy issues.

The system can use an optimized model to calculate the probability of formation for a series of antioxidant mixtures, generated by the system. The system can use an algorithm that can first randomly select components from the database of antioxidants and then gradually insert additional components (hydrogen bond donors and acceptors) from the DES/NADES database. While most of these mixtures will display low probability of formation, this approach can provide the widest versatility of combinations, that can still be interrogated in a few seconds. The system can provide a list of synergistic antioxidant combinations, ranked according to their probability to form ATX-DES (as exemplified in FIG. 12, for Ibuprofen). Considering that many of the most commonly used antioxidants are hydrophobic, this system can provide formation of DES/NADES with components featuring similar characteristics (e.g., standard deviation of logP<3), leading to solvents that could be easily added to fats and oils without affecting their stability.

In testing the system, one method of validating the system's predictions is by preparing the predicted ATX-DES by mild heating (80° C.) under magnetic stirring (350 RPM) for 2 h and then allowing it to cool down to room temperature. As a result, mixtures that remain clear and transparent for at least one week were designated as “stable”. Conversely, mixtures that exhibit precipitation or cloudiness in under one week were labeled as “non-stable” and were be abandoned. Out of the millions of possible combinations generated the focus can be on the combinations most likely to form stable DES/NADES. It is important to note that those mixtures formed with potentially toxic components (e.g. undecanoic acid) or with off-flavors (e.g., choline chloride) can be experimentally evaluated with the purpose of validating the predictions from the algorithm.

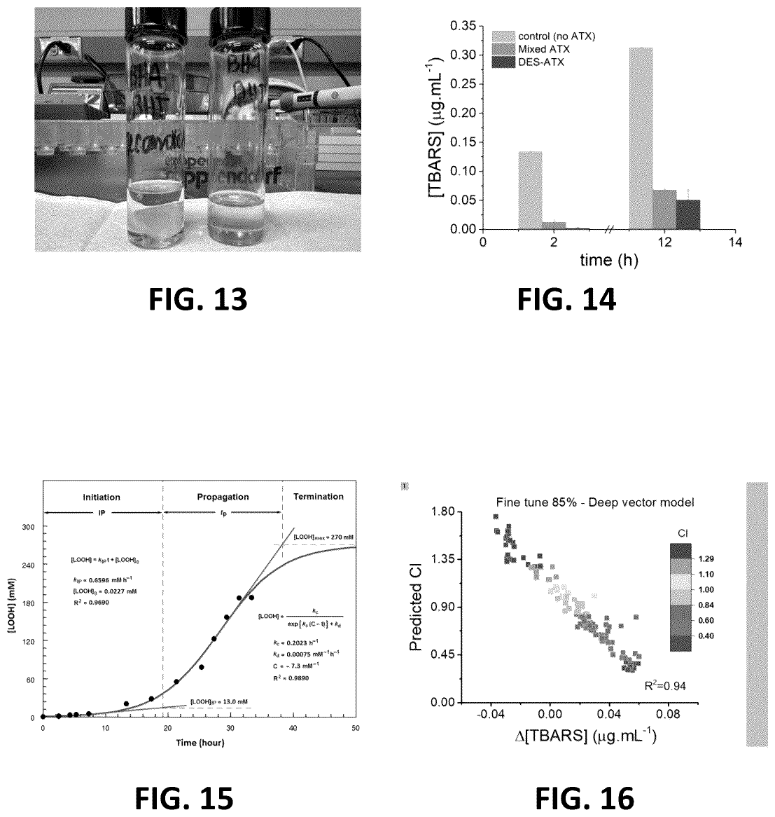

The system can provide an extensive database with examples of DES/NADES integrating synergistic mixtures of antioxidants, ranked by the probability of formation, and leading to a distribution similar to the one shown herein in FIG. 12 (calculated for ibuprofen). The system can provide at least several hundred combinations likely to form stable DES/NADES, out of which a selection (e.g., the top 50) can be used. Using a cut-off probability of 80%, the system could reasonably expect to obtain multiple DES-ATX that can be advanced to experimental testing. Depending on the stability results, the cut-off could be increased (targeting the uppermost stable mixtures) or decreased, leading to a larger number of samples. To help gauge the potential capabilities of the proposed approach, FIG. 13 shows two examples of ATX-DES predicted by the system and its algorithms. On the left is an equimolar mixture of BHA, BHT, and decanoic acid (low probability of formation), where a large crystal was formed in less than 24 hours (post synthesis) at room temperature. On the right is an equimolar mixture of BHA and BHT (high probability of formation predicted by the system), that remained in liquid form for at least 1 week and that represents a stable ATX-DES, underscoring the accuracy and reliability of the system. Moreover, this ATX-DES (FIG. 13, right vial) displayed significantly higher antioxidant capacity than the non-DES mixtures of the same antioxidants at the same concentrations (as shown in FIG. 14), and the individual antioxidants at the same concentrations.

The system can be validated by determining the antioxidant power of the synergistic mixtures identified herein. For these validations, real samples of fats (lard, tallow, chicken) and oils (soybean, rapeseed, olive) can be used. In this way, specific data and kinetics can be obtained related to the oxidation process. These results not only allow for the understanding of the effect of the sample type but also compare the oxidation process for bare samples with that of samples mixed with each of the selected antioxidants, their synergistic mixtures, and the DES-forms of the synergistic mixtures. For each sample, a profile similar to the one shown in FIG. 15 is typically obtained, where the time required to start the propagation phase can be inversely proportional with respect to the synergistic effect.

The multi-dimensional deep vector model will provide a database of mixtures of antioxidants, ranked according to their probability of displaying synergistic effects. These predictions (generated from fingerprints, vectors including selected features) can be more accurate than those obtained with the existing language-based model. As a sample of the improved capabilities of the proposed vector model, FIG. 16 displays the CI value (predicted with a non-optimized model) as a function of the experimental ΔTBARS. In one embedment, the power of the system can be determined by a comparison with those in FIG. 7D (transformer model), which can be a more advanced method. This embodiment may have overfitting issues that could arise (model memorizes the training dataset but underperforms on the evaluation dataset), and if so, the system can include more regularization (higher dropout rates), reduce the number of layers, change the network complexity (number/values of weights) or add a second fine-tuning loop using a fraction of the experimental data. It is also important to note that under this embodiment, the model will enable the consideration of the kinetics for the oxidation for specific samples under specific experimental conditions (ATX used, temperature, etc.). The system can also include a script to use the information available to calculate the amount of antioxidants needed to preserve samples for a predetermined period of time. This information can be used to predict and prevent rancidity under storage/transport conditions considering weather and traffic information.

The system can understand hydrogen bonding in the context of the formation of functional DES. However, if it is the case that the system does not recognize the structure and function of antioxidants, this issue may lead to inaccurate predictions. In this case, a similarity analysis can be performed that can focus on those antioxidants sharing structural characteristics with hydrogen bond donors/acceptors already in our DES database. The system allows the generation of several mixtures that can be experimentally assessed in the lab which can be used for specific tasks of creating compounds.

In one embodiment, the database can be augmented using RDKit, following the procedure described above. This process will generate the required vectorized representations of the antioxidants, that can be used by the system.

The optimized multi-dimensional deep vector model can be used to calculate the probability of formation for a series of antioxidant mixtures, generated by the system. The system can first randomly select components from the database of antioxidants and then gradually insert additional components (e.g., hydrogen bond donors and acceptors) from the DES database. This approach can provide the widest versatility of combinations, that can still be interrogated in a few seconds. At the end of this phase, we expect to obtain a list of synergistic antioxidant combinations, ranked according to their probability to form ATX-DES (as exemplified in FIG. 12, calculated for ibuprofen). Considering that many of the most commonly used antioxidants are hydrophobic, the system can provide DES/NADES with components featuring similar characteristics (e.g., hydrophobic behavior), leading to solvents that could be easily added to fats and oils without affecting their stability.

In one validation method, a determination is made of the antioxidant power of the synergistic mixtures. For these experiments, samples of fats (lard, tallow, chicken) and oils (soybean, rapeseed, olive) can be used. In this way, specific data and kinetics related to the oxidation process can be obtained. These results will not only allow an understanding of the effect of the sample type but also compare the oxidation process for bare samples with that of samples mixed with each of the selected antioxidants, their synergistic mixtures, and the DES-forms of the synergistic mixtures. Besides the controls, the system algorithm can be challenged experimentally by measuring the antioxidant power of some of the most synergistic mixtures (e.g., 20), at certain concentration levels (e.g., 3) and specifically addressing the kinetics of the process (data collected multiple times such as at least at 5 different times). For these experiments, the extent of lipid oxidation by the thiobarbituric acid reactive substances (TBARS) assay.

The system can obtain a profile like the one shown in FIG. 15, showing the time required to start the propagation phase, which is inversely proportional with respect to the antioxidant effect. This kinetic dependence has been reported for the oxidation of multiple samples. This analysis could not only greatly simplify the analysis of samples (as the corresponding data can fit with a similar function) but also enable assessing the results in terms of the kinetic parameters representing the accumulation of lipid hydroperoxides during the propagation phase of the oxidation.

The system can utilize functional DES to enhance antioxidant synergism in edible fats and oils. These functional DES may be formed by combining synergistic mixtures of antioxidant molecules as hydrogen bond donors/acceptors. In one implementation, the system identified mixtures of BHA (tert-butyl-4-hydroxyanisole) and BHT (3,5-di-tert-butyl-4-hydroxytoluene) as promising candidates for forming functional antioxidant DES. Experimental testing demonstrated that equimolar mixtures of BHA and BHT formed stable DES that remained liquid for over 4 weeks at room temperature. The antioxidant capacity of the BHA-BHT DES was evaluated using the thiobarbituric acid reactive substances (TBARS) assay on oleic acid samples. Results showed that the DES form exhibited significantly enhanced antioxidant activity compared to non-DES mixtures of the same antioxidants at equivalent concentrations. Specifically, the BHA-BHT DES reduced TBARS values by 56.7% compared to the control oleic acid sample, outperforming both individual antioxidants and their non-DES mixtures.

Further testing on commercial fat samples including olive oil, pork lard, and duck fat demonstrated the broad applicability of the functional DES approach. In olive oil, the BHA-BHT DES reduced TBARS values by 62% compared to the control, while in pork lard and duck fat, reductions of 86% and 68% were observed, respectively. In most all cases, the DES formulations showed superior antioxidant activity compared to non-DES mixtures of the same components.

The enhanced effectiveness of the functional DES may be attributed to the formation of a hydrogen bond network between the antioxidant molecules. Density functional theory (DFT) calculations suggested that the DES formation lowered the bond dissociation enthalpy of the antioxidants, potentially increasing their radical scavenging capacity. Kinetic analysis of the oxidation process in the presence of the functional DES showed an extended initiation phase and delayed onset of the propagation phase compared to controls. This indicates that the DES formulation may effectively inhibit the initial formation of lipid hydroperoxides and slow the chain reaction of lipid oxidation.

The system's ability to predict and formulate these synergistic antioxidant DES opens new possibilities for developing highly effective preservatives for fats and oils. The functional DES approach may provide a means to significantly enhance the oxidative stability and shelf life of lipid-containing food products while potentially reducing the overall quantity of antioxidants required.

As shown, the system may utilize various sequences in the application of algorithms to optimize the prediction and formulation of synergistic antioxidant mixtures and functional DES. In some aspects, the system can employ a flexible approach that allows for different algorithmic sequences to be explored and evaluated. One possible sequence may involve first identifying promising antioxidant combinations based on their predicted synergistic effects, followed by assessing their potential to form stable DES. In this approach, the system may initially screen a database of antioxidant compounds using machine learning algorithms to predict synergistic interactions. The most promising antioxidant combinations can then be evaluated for their likelihood of forming stable DES using separate predictive models that consider factors such as hydrogen bonding potential and physicochemical properties.

Alternatively, the system may reverse this sequence by first identifying potential DES formulations based on their predicted stability and desirable properties, and then evaluating their antioxidant potential. In this case, the initial focus may be on predicting stable DES combinations using molecular modeling and machine learning techniques. The resulting DES candidates can then be assessed for their potential antioxidant activity, either through computational methods or experimental testing.

In some implementations, the system may employ a parallel or iterative approach where both antioxidant synergy and DES formation potential are evaluated simultaneously or in alternating steps. This method may allow for continuous refinement of predictions as new data becomes available from both computational and experimental sources.

The system may also incorporate adaptive algorithms that can dynamically adjust the sequence based on intermediate results or user-defined priorities. For example, if initial screening identifies a particularly promising antioxidant combination, the system may prioritize exploring various DES formulations incorporating those specific antioxidants. By allowing for flexibility in the algorithmic sequence, the system may be capable of adapting to different research goals, available data, and computational resources. This versatility may enhance the system's ability to discover novel and effective antioxidant DES formulations across a wide range of potential applications.

In addition, various embodiments disclosed herein further relate to methods system and methods of training and using computer systems for analysis and prediction compounds that includes program code for performing various computer-implemented operations. The computer-readable medium is any data storage device that can store data which can thereafter be read by a computer system. Examples of computer-readable media include, but are not limited to, all the media mentioned above: magnetic media such as hard disks, floppy disks, and magnetic tape; optical media such as CD-ROM disks; magneto-optical media such as optical disks; and specially configured hardware devices such as application-specific integrated circuits, programmable logic devices, and ROM and RAM devices. Examples of program code include both machine code, as produced, for example, by a compiler, or files containing higher level code, for example, a script that can be executed using an interpreter.

The claimed invention advances the state of the art in computer technology, particularly in the field of predictive modeling for antioxidant interactions. By utilizing a multi-dimensional deep vector model to analyze and predict synergistic antioxidant combinations, the system significantly improves upon existing language-based models. This approach enables more accurate predictions of antioxidant behavior, as demonstrated by the improved correlation between predicted Combination Index (CI) values and experimental ΔTBARS measurements. The system's ability to process millions of potential combinations and rank them according to their probability of forming stable DES/NADES mixtures represents a substantial improvement over the traditional trial-and-error method. Furthermore, the incorporation of kinetic data and environmental factors into the predictive model allows for more comprehensive and context-specific predictions, enhancing the practical applicability of the technology in real-world scenarios such as food preservation and storage optimization.

Various examples of embodiments described herein are described in connection with flow diagrams. Although the examples may include certain steps performed in a particular order, according to various embodiments, various steps may be performed in various orders and/or various steps may be combined into a single step or in parallel.

Although the foregoing embodiments have been described in some detail for purposes of clarity of understanding, it is understood that the above descriptions and illustrations are intended to be illustrative and not restrictive. Other embodiments as well as many applications besides the examples provided will be apparent to those of skill in the art upon reading the above description. The scope of the invention should, therefore, be determined not with reference to the above description, but should instead be determined with reference to the appended claims, along with the full scope of equivalents to which such claims are entitled. The omission in the following claims of any aspect of subject matter that is disclosed herein is not a disclaimer of such subject matter, nor should it be regarded that the inventor did not consider such subject matter to be part of the disclosed inventive subject matter.

A number of implementations have been described. Nevertheless, it will be understood that various modifications may be made without departing from the spirit and scope of the disclosure. Accordingly, other implementations are within the scope of the following claims.

What is claimed is:

1. A computerized system comprising:
a pre-trained artificial neural network model trained using general unlabeled chemical data included in a computer system wherein the computer system is adapted to:
fine-tune the artificial neural network model using a set of deep eutectic solvent (DES) and natural deep eutectic solvent (NADES) mixtures, wherein the set includes stable DES/NADES mixtures, mixtures that do not form DES/NADES, and DES/NADES mixtures that are not stable;
receive a first dataset of potential antioxidant mixtures;
create a predictive model based on the first dataset;
enhance the predictive model using an enhancement dataset;
predict an antioxidant mixture using the enhanced predictive model; and
display the predicted antioxidant mixture.

2. The computerized system of claim 1, wherein the computer system is further adapted to use a data augmentation method to overcome overfitting.

3. The computerized system of claim 2, wherein the data augmentation method comprises representing stoichiometric ratios as repetitions of antioxidant compounds in SMILES notation.

4. The computerized system of claim 1, wherein the set of deep eutectic solvents is a set of functional deep eutectic solvents.

5. The computerized system of claim 1, wherein the computer system is further adapted to adjust weights and biases of the artificial neural network model during training to improve performance on unseen chemical data.

6. The computerized system of claim 1, wherein the computer system is further adapted to perform a second fine-tuning step using increasing amounts of experimental data.

7. The computerized system of claim 1, wherein the predicted antioxidant mixture is a functional DES integrating synergistic mixtures of antioxidants.

8. A computerized system comprising:
an artificial neural network;
a set of antioxidant regressors;
a computer device configured to:
select an antioxidant regressor from the set of antioxidant regressors according to a performance assessment;
fine-tune the selected antioxidant regressor with benchtop data;
blend the fine-tuned antioxidant regressor with experimental chemistry data;
associate antioxidant mixtures with combination index values; and
predict synergistic antioxidant mixtures according to the artificial neural network and based on the associated combination index values.

9. The computerized system of claim 8, wherein the computer device is further configured to use molecular fingerprints and chemical descriptors to predict synergistic mixtures of antioxidants.

10. The computerized system of claim 9, wherein the molecular fingerprints are derived from molecular graphs and enable calculations based on global molecular descriptors.

11. The computerized system of claim 10, wherein the processor is further configured to augment a database using a cheminformatics toolkit to generate vectorized representations of antioxidants.

12. The computerized system of claim 11, wherein the processor is further configured to select potentially relevant chemical descriptors including number of atoms, number of heavy atoms, polar surface area, molecular weight, number of aromatic rings, number of heteroatoms, logP, number of carbon atoms, number of oxygen atoms, number of nitrogen atoms, and number of chloride atoms.

13. The computerized system of claim 12, wherein the processor is further configured to query hydrogen bond donor count and hydrogen bond acceptor count.

14. The computerized system of claim 13, wherein the processor is further configured to build feature maps that indicate a degree of molecular overlap between selected structures.

15. A computerized process comprising:
receiving an initial database of potential antioxidant mixtures;
dividing the initial database into a training portion and a testing portion, wherein the testing portion includes textual and numerical representations;
training a machine learning model using the training portion;
evaluating the trained model using the testing portion;
fine-tuning the model based on the evaluation;
generating a confidence index for predicted antioxidant mixtures; and
outputting predicted antioxidant mixtures with associated confidence indices.

16. The computerized process of claim 15, wherein the initial database comprises historical data on antioxidant effectiveness in various compositions.

17. The computerized process of claim 16, wherein the textual representations in the testing portion use Simplified Molecular Input Line Entry System (SMILES) notation to represent antioxidant combinations.

18. The computerized process of claim 17, wherein the numerical representations in the testing portion use stoichiometric ratios as repetitions of a same antioxidant compound to avoid overfitting.

19. The computerized process of claim 18, further comprising ranking predicted antioxidant mixtures according to a time required to start a propagation phase in an oxidation process.

20. The computerized process of claim 19, further comprising determining a number of antioxidant mixtures to create according to stability results of previously predicted mixtures.
